(19) United States
(12) Patent Application Publication
(10) Pub. No.: US 2014/0090108 A1
(43) Pub. Date: Mar. 27, 2014

(54) Vectors and Methods For Enhancing Recombinant Protein Expression in Plants

(71) Applicants: Freydoun Garabagi, Guelph (CA); Michael D. McLean, Guelph (CA); J. Christopher Hall, Guelph (CA)

(72) Inventors: Freydoun Garabagi, Guelph (CA); Michael D. McLean, Guelph (CA); J. Christopher Hall, Guelph (CA)

(21) Appl. No.: 13/837,612

(22) Filed: Mar. 15, 2013

Related U.S. Application Data
(60) Provisional application No. 61/702,395, filed on Sep. 18, 2012.

(51) Int. Cl. C12N 15/82

(52) U.S. Cl. CPC C12N 15/8257
USPC 800/288; 435/320.1; 435/69.6; 435/468; 435/69.1

(57) ABSTRACT

Expression vectors and methods of their use for enhancing the production of recombinant proteins in plants or plant cells are described. Production can be further enhanced upon co-expression of the P19 suppressor of gene-silencing from tomato bushy stunt virus. Preferably, the recombinant proteins are therapeutic enzymes and/or antibodies and methods are carried out in Nicotiana benthamiana—optionally an RNAi-based glycomodified strain—or in the Nicotiana tabacum cultivar Little Crittenden.

This application claims the benefit under 35 USC §119(e) from U.S. Provisional patent application Ser. No. 61/702,395, filed Sep. 18, 2012, which is incorporated herein by reference.

### INCORPORATION OF SEQUENCE LISTING

A computer readable form of the Sequence Listing “20436-P41839US01_SequenceListing.txt” (8,192), submitted via EFS-WEB and created on Mar. 14, 2013 is herein incorporated by reference.

### FIELD OF THE DISCLOSURE

The present application relates to a set of expression vectors designed for enhancing the production of recombinant proteins in plants and methods of using same.

### BACKGROUND OF THE DISCLOSURE

The gene-silencing machinery of plants is involved in regulating expression of endogenous gene transcripts as well as reducing or eliminating the effects of invading pathogens such as viruses (Baulcombe, 2004; Reinhart et al., 2002). As a countermeasure to this defense mechanism, viruses encode for proteins that act as suppressors of gene-silencing (SGS). P19 from the Tomato Bushy Stunt Virus (TBSV) is an example of proteins known to function as a potent suppressor of gene-silencing in plants as well as in animals (Scholthof, 2006; Voinnet et al., 1999). Plants also react to most transfer DNA (T-DNA) transgenes that invade their genomes by initiating a post-transcriptional gene silencing response (Baulcombe, 2004; Brodersen and Voinnet, 2006). The inhibitory effect of P19 on the gene-silencing pathway has been exploited to enhance expression levels of recombinant proteins in plants (Voinnet et al., 2003), but its use has been limited to transient expression only, mainly due to the deleterious effects of this protein when expressed constitutively at high levels in a transgenic setting (Siddiqui et al., 2008).

There are two main cellular gene-silencing mechanisms in plants, the small interfering RNA (siRNA) and the micro RNA (miRNA) silencing pathways (Carthew and Sontheimer, 2009), which are collectively referred to as interfering RNA (RNAi). The two systems show a great deal of similarity in their mechanisms of action, as they share some key enzymes (Brodersen and Voinnet, 2006). Both systems identify their target nucleic acid (viral RNA, viral DNA, transgene mRNA, endogenous mRNA) by a complex known as RNA-induced silencing complex (RISC). RISC carries a complementary single stranded RNA probe for its target, which upon binding, is either blocked or degraded.

The mechanism of action of P19 in suppressing gene-silencing at the molecular level has become better understood in recent times (Burgyan et al., 2004), but certain aspects still remain unclear. P19 is a multifunctional protein that is active as a dimer and found in both the cytosol and the nucleus (Park et al., 2004). It is capable of binding siRNA and miRNA molecules in a non-specific fashion (Dunoyer et al., 2004). Since there is a rise in virus-derived siRNA levels in plants in response to infection (Scholthof et al., 1995), P19 acts to reduce the amount of free siRNA duplexes through non-specific binding and represses the silencing response by interfering with siRNA loading of RISC (Hsieh et al., 2009). Studies on TBSV mutants with lowered levels of P19 have shown that a high titer of the protein is critical for exerting its biological activity (Qiu et al., 2002; Scholthof et al., 1999). Despite P19's non-specific siRNA binding, the effects brought about by this protein show host-specificity (Ahn et al., 2011; Angel et al., 2011; Siddiqui et al., 2008).

Nicotiana species display a variation in induction of the hypersensitive response (HR) to the P19 protein of TBSV, which is indicated by an initial leaf discoloration that leads to necrosis at the site of infection (Angel et al., 2011). In N. tabacum cv. Samsun, discoloration from HR develops 2-3 days after infiltration of leaves with P19, leading to fully dehydrated spots on day 7, while the same treatment yields no necrosis or discoloration in N. benthamiana. Stable transgenic expression of P19, however, does not elicit HR in either N. tabacum cv. Xanthi or N. benthamiana, indicating that high titers of P19 are required for triggering this response (Siddiqui et al., 2008). The difference in the HR generated in N. tabacum and N. benthamiana in response to P19 has been attributed to a host protein that is the product of a putative resistance (R) gene (Jovel et al., 2011). The putative R gene product is thought to identify P19 and trigger a cascade of events that lead to local necrosis for slowing down the spread of virus. Although the R gene product that specifically interacts with P19 has not been identified, experiments show that the resistance conferred by this gene product is inherited in a dominant fashion (Jovel et al., 2011).

The extent to which P19 increases expression seems to vary for different recombinant proteins. Several reports indicate that the expression of Green Fluorescent Protein (GFP), a commonly used reporter, is boosted approximately 50-fold when co-expressed with P19 (Voinnet et al., 2003; Zheng et al., 2009). Expression of antibodies and other therapeutic proteins, on the other hand, have only been enhanced by five-fold (Saxena et al., 2011; Zheng et al., 2009).

In the context of plant-derived therapeutic proteins, another very important consideration is their glycan profile, since this post-translational modification impacts the efficacy of therapeutic proteins and can be a major factor in batch-to-batch variability of recombinant therapeutic proteins (Gomord et al., 2010; Schiestl et al., 2011). Plant-specific sugar residues on the N-glycan core, namely core α1,3-fucose and β1,2-xylose, are immunogenic in mammals (Bardor et al., 2003; Jin et al., 2008). As a result, a great deal of effort has been directed towards creating plants with modified humanized glycosylation patterns (Cox et al., 2006; Sourrouille et al., 2008; Strasser et al., 2008). For the most part, glycomodified plants have been created through RNAi gene-silencing technology, mainly due to the existence of multiple endogenous fucosyltransferase and xylosyltransferase genes in most plants (Cox et al., 2006; Sourrouille et al., 2008; Strasser et al., 2008). Consequently, interference in the siRNA pathway by P19 becomes a concern when RNAi-generated genetic backgrounds are to be used as expression hosts for producing therapeutic proteins, especially since in an unrelated case, P19 was shown to repress the knockdown of a previously established RNAi transgenic line (Ahn et al., 2011).

### SUMMARY OF THE DISCLOSURE

The present inventors have designed and tested a suite of plant expression vectors which are suitable for enhancing expression of recombinant protein in both transient expression and stable transgenic plants. The unique combination of promoter, 5′ UTR, and 3′ UTR/terminator in these vectors drives high levels of heterologous protein expression in plants, including Nicotiana benthamiana and Nicotiana tabacum.

Accordingly, the present application provides an expression vector comprising:

(a) a promoter selected from (i) the 35S promoter of the Cauliflower Mosaic Virus (CaMV) or (ii) the promoter of the ribulose bisphosphate carboxylase (rbc) small subunit gene of Chrysanthemum morifolium;

(b) a 5′ untranslated region (UTR) selected from (i) the 35S 5′ UTR of CaMV or (ii) the 5′ UTR of the rbc small subunit gene of C. morifolium; and

(c) a 3′ UTR and terminator sequence selected from (i) the 3′ UTR and terminator sequence of the nopaline synthase (nos) gene of Agrobacterium, (ii) the 3′ UTR and terminator sequence of the osmotin (osm) gene of Oryza sativa, (iii) the 3′ UTR and terminator sequence from the rbc small subunit gene of C. morifolium or (iv) a truncated version, by 162 bp as defined by a BspEI recognition site, of the 3′ UTR and terminator sequence from the rbc small subunit gene of C. morifolium.

In one embodiment, a nucleic acid sequence encoding a recombinant protein is cloned in the above-mentioned vectors.

The present application further provides a method of enhancing the production of a recombinant protein in a plant comprising:

(i) introducing an expression vector comprising
(a) a promoter selected from (i) the 35S promoter of the Cauliflower Mosaic Virus (CaMV) or (ii) the promoter of the ribulose bisphosphate carboxylase (rbc) small subunit gene of Chrysanthemum morifolium;
(b) a 5′ untranslated region (UTR) selected from (i) the 35S 5′ UTR of CaMV or (ii) the 5′ UTR of the rbc small subunit gene of C. morifolium;
(c) a 3′ UTR and terminator sequence selected from (i) the 3′ UTR and terminator sequence of the nopaline synthase (nos) gene of Agrobacterium, (ii) the 3′ UTR and terminator sequence of the osmotin (osm) gene of Oryza sativa, (iii) the 3′ UTR and terminator sequence from the rbc small subunit gene of C. morifolium or (iv) a truncated version, by 162 bp as defined by a BspEI recognition site, of the 3′ UTR and terminator sequence from the rbc small subunit gene of C. morifolium; and
(d) a nucleic acid sequence encoding a recombinant protein into a plant or plant cell; and

(ii) growing the plant or plant cell to obtain a plant that expresses the recombinant protein.

In one embodiment, the recombinant protein is co-expressed with the P19 suppressor of gene-silencing protein from tomato bushy stunt virus (TBSV).

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1: Diagram of the expression cassettes used in Example 1. The expression cassettes shown here were situated on the T-DNA region of binary vectors. Vector 102mAb was the only vector carrying the heavy (HC) and light chains (LC) of trastuzumab on the same T-DNA. When expressing trastuzumab with vectors 103mAb-106mAb, the HC and LC were co-expressed to produce fully assembled IgG molecules.

FIG. 2: Western blot analysis of trastuzumab expressed transiently with different plant expression vectors in N. benthamiana. Expression of the 103-106mAb vectors was analyzed over 6 days. Plants were treated by vacuum infiltration. Each lane represents a pooled sample, created by mixing three leaf samples. The vectors were either expressed alone (A), or together with P19 (B). All vector sets carried the same codes for the HC and LC of trastuzumab coupled with different UTRs. Different expression dynamics were observed when vectors were expressed alone or together with P19, as determined by ELISA (C).

FIG. 3: Western blot analysis of trastuzumab expressed transiently with (A) 102mAb and with (B) TMV/PVX (virus-based) expression vectors in N. benthamiana. Plants were treated by spot infiltration. Pooled samples were generated by combining three infiltrated spots. Two pooled samples (harvested 5 d.p.i.) are shown for each treatment. Similar to 106mAb, co-expression of P19 did not affect the level of trastuzumab expressed with either vector.

FIG. 4: Western blot analysis showing the dose-dependent effect of P19 on enhancing recombinant antibody expression in N. benthamiana. 103mAb vectors were co-expressed with P19 at three different concentrations of Agrobacterium, OD₆₀₀=0.2, 0.02, and 0.002. Plants were treated by spot infiltration. Pooled sampled were generated by combining three infiltrated spots. Each lane represents a pooled sample. The boosting effect of P19 was most prominent when applied at the higher concentration, regardless of the concentration of 103mAb. P19 had no boosting effect on antibody expression when applied at OD₆₀₀=0.002.

FIG. 5: Differential response of N. tabacum and N. benthamiana to P19. Western blot analysis showing transient expression of trastuzumab alone or together with P19 in N. benthamiana and five different N. tabacum cultivars (A) and N. tabacum crosses (B). Plants were treated by spot infiltration. Samples were pooled by combining three infiltrated spots. Each lane represents a pooled sample. Co-expression of 103mAb with P19 resulted in a significant reduction in antibody expression in all tobacco cultivars except in LCR. The drop in antibody expression indicates an intensified state of RNAi silencing. Crosses between N. tabacum I-64 and LCR, and N. benthamiana and N. tabacum I-64 (NBT) showed a similar drop in antibody expression when P19 was co-expressed with 103mAb (B). All Nicotiana species that showed a drop in antibody expression in the presence of P19 also displayed discoloration at the site of infection, which lead to necrosis about day 3 days post-infection (C). Images here show infiltrated spots at 5 days post-infection. XAN, N. tabacum cv. Xanthi; PH, N. tabacum cv. Petite Havana H4; LCR, N. tabacum cv. Little Crittenden; BEN, N. benthamiana; NBT, N. benthamian×N. tabacum cv. 1-64.

FIG. 6: N-Glycan profiles of 103mAb expressed in N. benthamiana WT (A) and in ΔXTFT without (B) and with P19 (C). N-glycan analyses were carried out by liquid-chromatography-electrospray ionization-mass spectrometry (LC-ESI-MS) of tryptic glycopeptides as described previously (Stadlmann et al., 2008; Strasser et al., 2008). Note, that incomplete tryptic digest results in the generation of two glycopeptides that differ by 482 Da. Glycopeptide 1 is indicated with asterisks (*). See http://www.proglycan.com for N-glycan abbreviations.

FIG. 7: Transient co-infiltration of P19 expression vector greatly enhances the expression of two more therapeutic antibodies in Nicotiana benthamiana. Coding sequences for both anti-HIV mAb 4E10 and bevacizumab were spot-infiltrated, with and without P19, into N. benthamiana leaf tissue and harvested after 7 days. Each Agrobacterium strain was applied at a final OD₆₀₀ of 0.2. Tissue harvests for each treatment included 3 or 4 spots, which were pooled and total soluble protein (TSP) was extracted as described in Garabagi et al. (2012a,b). A 10% non-reducing SDS-PAGE gel was run that included 10 μg TSP for each plant sample. Electrophoretically separated proteins were subsequently electrotransferred to PVDF membrane and probed with combined anti-γ and anti-κ antibody probes conjugated to alkaline phosphatase (also described in Garabagi et al., 2012a). Results indicate that co-expression of P19 greatly enhances expression of both antibodies irrespective of antibody expression vector. The gel loading scheme is tabulated above the immunoblot image, and the size of each molecular weight marker band in lane 1 is given on the left in kDa. The human serum immunoglobulin quantification standard in lane 10 is 500 ng. mAb=monoclonal antibody; HC=heavy chain vector; LC=light chain vector. Note that vector p105T contains a truncated 3′UTR compared with the original p105 vector, with 162 less base pairs (bps) due to a BspEI-BspEI deletion.

FIG. 8: Transient expression of human butyrylcholinesterase (BChE) in N. benthamiana is greatly enhanced by co-expression of P19. Two different BChE expression vectors were constructed in p105T: (1) with the Arabidopsis basic chitinase (abc) signal sequence and a synthetic BChE coding sequence optimized for expression in plants referred to as E2, i.e., abc-BChE2; and (2) with the native human BChE signal sequence (hSS) and another synthetic BChE coding sequence optimized for expression in plants referred to as E3, hSS-BChE3. These were introduced into whole N. benthamiana plants by vacuum infiltration (Garabagi et al., 2012a,b), either with or without the P19 expression vector described in this application. Samples were taken at 5, 7 and 9 days post-infiltration (DPI) and BChE activity was measured by Ellman Assay (Ellman et al., 1961). All histogram bars present amounts of BChE in mg BChE/kg leaf tissue, by converting activity measurements to mass of enzyme using the specific activity conversion factor of 718.3 activity units per mg of BChE determined in Weber et al. (2010). Note that background readings (as determined with untreated plant extracts) have been subtracted for the presentation of these data. Histogram bars indicate the mean BChE activity measured in 2 samples from 2 plants (4 total repeats each) with associated standard error bars.

FIG. 9: Diagram of one of the expression cassettes used in FIG. 7 and in FIG. 8. The 105 mAb expression cassette from FIG. 1 is shown at the top of the figure. Insertion of a coding sequence for a recombinant protein to be expressed in plants is performed by using the restriction endonuclease BspEI for ligation of the 3′ end of that coding sequence, resulting in a 162 bp deletion of the 5′ end of the Rbc 3′ UTR and terminator. The resulting 105T (T=truncated) expression cassette is shown at the bottom of the figure.

### DETAILED DESCRIPTION OF THE DISCLOSURE

As previously described, the present inventors have designed and tested a suite of plant expression vectors which are suitable for enhancing expression of recombinant protein in plants. The unique combination of promoter, 5′ UTR, and 3′ UTR/terminator in these vectors drives high levels of heterologous protein expression in plants, including Nicotiana benthamiana and Nicotiana tabacum.

### I. Expression Vectors

In one embodiment, the application provides an expression vector comprising:

(a) a promoter selected from (i) the 35S promoter of the Cauliflower Mosaic Virus (CaMV) or (ii) the promoter of the ribulose bisphosphate carboxylase (rbc) small subunit gene of Chrysanthemum morifolium;

(b) a 5′ untranslated region (UTR) selected from (i) the 35S 5′ UTR of CaMV or (ii) the 5′ UTR of the rbc small subunit gene of C. morifolium; and

(c) a 3′ UTR and terminator sequence selected from (i) the 3′ UTR and terminator sequence of the nopaline synthase (nos) gene of Agrobacterium, (ii) the 3′ UTR and terminator sequence of the osmotin (osm) gene of Oryza sativa, (iii) the 3′ UTR and terminator sequence from the rbc small subunit gene of C. morifolium or (iv) a truncated version, by 162 bp as defined by a BspEI recognition site, of the 3′ UTR and terminator sequence from the rbc small subunit gene of C. morifolium.

As used herein, the term “expression vector” means a nucleic acid molecule, such as a plasmid, comprising regulatory elements and a site for introducing transgenic DNA, which is used to introduce said transgenic DNA into a host cell. The transgenic DNA can encode a heterologous protein, which can be expressed in and isolated from plant cells.

Regulatory elements include promoters, 5′ and 3′ untranslated regions (UTRs) and terminator sequences or truncations thereof. The regulatory elements of the present invention can be selected from the 35S promoter and 5′UTR of the Cauliflower Mosaic Virus (CaMV; Genbank accession: AF140604), the promoter and 5′ UTR of ribulose bisphosphate carboxylase (rbc) small subunit gene from Chrysanthemum morifolium (Genbank accession: AY163904.1), the heat-shock (Hsp81.1) promoter from Arabidopsis thaliana, the 3′ UTR and terminator sequences from the nopaline synthase (nos) gene of Agrobacterium (Genbank accession: V00087.1), the 3′ UTR and terminator sequences from the osmotin (osm) gene of Oryza sativa (Genbank accession: L76377.1) and the 3′ UTR and terminator sequences from the rbc gene of C. morifolium. In one embodiment, the Hsp81.1 promoter from Arabidopsis is placed directly upstream of one of the other promoters.

In one embodiment, the expression vector comprises the 35S promoter of CaMV, operably linked to the 35S 5′ UTR of CaMV and the 3′ UTR and terminator sequence of the nos gene of Agrobacterium. This expression vector may also be referred to as p103.

In another embodiment, the expression vector comprises the 35S promoter of CaMV, operably linked to the 35S 5′ UTR of CaMV and the 3′ UTR and terminator sequence of the osm gene of Oryza sativa. This expression vector may also be referred to as p104.

In another embodiment, the expression vector comprises the 35S promoter of CaMV, operably linked to the 35S 5′ UTR of CaMV and the 3′ UTR and terminator sequence of the rbc small subunit gene of C. morifolium. This expression vector may also be referred to as p105.

In another embodiment, the expression vector comprises the 35S promoter of CaMV, operably linked to the 35S 5′ UTR of CaMV and a truncated version, by 162 bp as defined by a BspEI recognition site, of the 3′ UTR and terminator sequence from the rbc small subunit gene of C. morifolium. This expression vector may also be referred to as p105T.

In another embodiment, the expression vector comprises the promoter of the rbc small subunit gene of C. morifolium, operably linked to the 5′ UTR of the rbc small subunit gene of C. morifolium and the 3′ UTR and terminator sequence of the rbc small subunit gene of C. morifolium. This expression vector may also be referred to as p106.

As used herein, the term “nucleic acid molecule” means a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

In one embodiment, the application provides an expression vector comprising:

(a) a promoter selected from (i) the 35S promoter of the Cauliflower Mosaic Virus (CaMV) or (ii) the promoter of the ribulose bisphosphate carboxylase (rbc) small subunit gene of Chrysanthemum morifolium;

(b) a 5′ untranslated region (UTR) selected from (i) the 35S 5′ UTR of CaMV or (ii) the 5′ UTR of the rbc small subunit gene of C. morifolium;

(c) a 3′ UTR and terminator sequence selected from (i) the 3′ UTR and terminator sequence of the nopaline synthase (nos) gene of Agrobacterium, (ii) the 3′ UTR and terminator sequence of the osmotin (osm) gene of Oryza sativa, (iii) the 3′ UTR and terminator sequence from the rbc small subunit gene of C. morifolium, or (iv) a truncated version, by 162 bp as defined by a BspEI recognition site, of the 3′ UTR and terminator sequence from the rbc small subunit gene of C. morifolium and

(d) a nucleic acid sequence encoding a recombinant protein.

As used herein, the term “recombinant protein” means any polypeptide that can be expressed in a plant cell, wherein said polypeptide is encoded by transgenic DNA introduced into the plant cell via use of an expression vector. In a preferred embodiment, the expression vector is p103. In another preferred embodiment, the expression vector is p105 or p105T.

In one embodiment, the recombinant protein is an antibody or antibody fragment. In a specific embodiment, the antibody is trastuzumab or a modified form thereof, consisting of 2 heavy chains (HC) and 2 light chains (LC). Trastuzumab (Herceptin® Genentech Inc., San Francisco, Calif.) is a humanized murine immunoglobulin G1κ antibody that is used in the treatment of metastatic breast cancer.

In another specific embodiment, the antibody is bevacizumab or a modified form thereof, consisting of 2 heavy chains (HC) and 2 light chains (LC). Bevacizumab (trade name Avastin, Genentech/Roche) is an angiogenesis inhibitor, a drug that slows the growth of new blood vessels. It is licensed to treat various cancers, including colorectal, lung, breast, glioblastoma, kidney and ovarian.

In another specific embodiment, the recombinant protein is an enzyme such as a therapeutic enzyme. In a specific embodiment, the therapeutic enzyme is butyrylcholinesterase. Butyrylcholinesterase (also known as pseudocholinesterase, plasma cholinesterase, BCHE, or BuChE) is a non-specific cholinesterase enzyme that hydrolyses many different choline esters. In humans, it is found primarily in the liver and is encoded by the BCHE gene. It is being developed as an antidote to nerve-gas poisoning.

The nucleic acid molecules encoding the HC and LC of an antibody or antibody fragment or the coding sequence of a therapeutic enzyme can be incorporated separately into one expression vector each or incorporated together into a single expression vector comprising multiple expression cassettes.

As used herein, the term “expression cassette” means a single, operably linked set of regulatory elements that includes a promoter, a 5′ UTR, an insertion site for transgenic DNA, a 3′ UTR and a terminator sequence.

As used herein, the term “antibody fragment” includes, without limitation, Fab, Fab′, F(ab′)₂, scFv, dsFv, ds-scFv, dimers, minibodies, diabodies, and multimers thereof and bispecific antibody fragments.

In one embodiment, a signal peptide that directs the polypeptide to the secretory pathway of plant cells may be placed at the amino termini of recombinant proteins, including antibody HCs and/or LCs. In a specific embodiment, the Arabidopsis thaliana basic chitinase signal peptide (SP), namely MAKTNLFLFLIFSLLLSLSSA (SEQ ID NO:2), is placed at the amino-(N-)termini of both the HC and LC (Samac et al., 1990).

In another embodiment, the native human butyrylcholinesterase signal peptide (SP), namely MHSKVTIICIRFLFWFLLLCMLIGKSHT (SEQ ID NO:3), is placed at the amino-(N-)termini of a therapeutic enzyme such as butyrylcholinesterase (GenBank: AAA99296.1).

Other signal peptides can be mined from GenBank [http://www.ncbi.nlm.nih.gov/genbank/] or other such databases, and their sequences added to the N-termini of the HC or LC, nucleotides sequences for these being optimized for plant preferred codons as described above and then synthesized. The functionality of a SP sequence can be predicted using online freeware such as the SignalP program [http://www.cbs.dtu.dk/services/SignalP/].

In a specific embodiment, the nucleic acid constructs encoding recombinant proteins, including antibody HCs and/or LCs, and therapeutic proteins such as enzymes, are optimized for plant codon usage. In particular, the nucleic acid sequence encoding the heavy chain and light chain can be modified to incorporate preferred plant codons. In a specific embodiment, coding sequences for both the HC and LC, including the SP in both cases, were optimized for expression in Nicotiana species. The first goal of this procedure was to make the coding sequences more similar to those of Nicotiana species. Codon optimizations were performed utilizing online freeware, i.e., the Protein Back Translation program (Entelchon), and Nicotiana coding sequence preferences. Codons with the highest frequencies for each amino acid in Nicotiana species (Nakamura, 2005) were thereby incorporated. Furthermore, potential intervening sequence splice-site acceptor and donor motifs were identified (Shapiro et al., 1987; CNR National Research Council) and subsequently removed by replacement with nucleotides that resulted in codons encoding the same amino acids. Inverted repeat sequences were analyzed using the Genebee RNA Secondary Structure software package (Brodsky et al.; GeneBee Molecular Biology Server); nucleotides were changed to reduce the free energy (kilocalories per mole) of potential secondary structure while maintaining the polypeptide sequence. Likewise, repeated elements were analyzed (CNR National Research Council) and replaced where present. Potential methylation sites (i.e., CXG and CpG; Gardiner-Garden et al.) were replaced where possible and always without changing the encoded amino acid sequence. A Kozak (Kozak, 1984) optimized translation start site was engineered. Plant polyadenylation sites (i.e., AATAAA, AATGAA, AAATGGAAA, and AATGGAAATG; Li et al.; Rothnie) and ATTTA RNA instability elements (Ohme-Takagi et al.) were likewise avoided.

Seletectable marker genes can also be linked on the T-DNA, such as kanamycin resistance gene (also known as neomycin phonphatase gene II, or nptII), Basta resistance gene, hygromycin resistance gene, or others.

### II. P19 Suppressor of Gene-Silencing

In one embodiment the recombinant protein, such as an antibody or therapeutic enzyme, is co-expressed with the P19 protein from Tomato Bushy Stunt Virus (TBSV; Genbank accession: M21958). In a preferred embodiment, the P19 protein from TBSV is expressed from a nucleic acid molecule which has been modified to optimize expression levels in tobacco plants. In a specific embodiment, the modified P19-encoding nucleic acid molecule has the sequence shown in SEQ ID NO:1.

In one embodiment, the P19-encoding nucleic acid is incorporated into one of the expression vectors of the present invention. In a preferred embodiment, the expression vector is p103, p105 or p105T.

The P19 protein can be expressed from an expression vector comprising a single expression cassette or from an expression vector containing one or more additional cassettes, wherein the one or more additional cassettes comprise transgenic DNA encoding one or more recombinant proteins.

### III. Method of Plant Transformation

The inventors have demonstrated that they can achieve high levels of expression of recombinant proteins using the vectors described herein.

Accordingly, the present application provides a method of enhancing the production of a recombinant protein in a plant comprising:

(i) introducing an expression vector comprising
(a) a promoter selected from (i) the 35S promoter of the Cauliflower Mosaic Virus (CaMV) or (ii) the promoter of the ribulose bisphosphate carboxylase (rbc) small subunit gene of Chrysanthemum morifolium;
(b) a 5′ untranslated region (UTR) selected from (i) the 35S 5′ UTR of CaMV or (ii) the 5′ UTR of the rbc small subunit gene of C. morifolium;
(c) a 3′ UTR and terminator sequence selected from (i) the 3′ UTR and terminator sequence of the nopaline synthase (nos) gene of Agrobacterium, (ii) the 3′ UTR and terminator sequence of the osmotin (osm) gene of Oryza sativa, (iii) the 3′ UTR and terminator sequence from the rbc small subunit gene of C. morifolium, or (iv) a truncated version, by 162 bp as defined by a BspEI recognition site, of the 3′ UTR and terminator sequence from the rbc small subunit gene of C. morifolium; and
(d) a nucleic acid sequence encoding a recombinant protein into a plant or plant cell; and

(ii) growing the plant or plant cell to obtain a plant that expresses the recombinant protein.

In one embodiment, the recombinant protein is the only heterologous protein expressed in the plant or plant cell. In a preferred embodiment, the recombinant protein is co-expressed with the P19 protein from TBSV. The P19 protein and expression vectors for expressing it are described above.

In one embodiment, the recombinant protein is an antibody or antibody fragment, comprising a heavy chain variable region and a light chain variable region. In a specific embodiment, the antibody is trastuzumab. In another specific embodiment, the antibody is bevacizumab.

In another specific embodiment, the recombinant protein is an enzyme such as butyrylcholinesterase.

In one embodiment, the nucleic acid molecule encoding the heavy chain variable region and the nucleic acid molecule encoding the light chain variable region may be introduced into the plant cell on separate expression vectors. In another embodiment, the nucleic acid molecule encoding the heavy chain variable region and the nucleic acid molecule encoding the light chain variable region may be introduced into the plant cell on the same expression vector. In such an embodiment, the heavy chain and the light chain would be expressed separately and then combine in the plant cell in order to prepare the desired antibody or antibody fragment.

The phrase “introducing” an expression vector into a plant or plant cell” includes both the stable integration of the recombinant nucleic acid molecule into the genome of a plant cell to prepare a transgenic plant as well as the transient integration of the recombinant nucleic acid into a plant or part thereof.

The expression vectors may be introduced into the plant cell using techniques known in the art including, without limitation, electroporation, an accelerated particle delivery method, a cell fusion method or by any other method to deliver the expression vectors to a plant cell, including Agrobacterium mediated delivery, or other bacterial delivery such as Rhizobium sp. NGR234, Sinorhizobium meliloti and Mesorhizobium loti (Chung et al, 2006).

The plant cell may be any plant cell, including, without limitation, tobacco plants, tomato plants, maize plants, alfalfa plants, Nicotiana benthamiana, Nicotiana tabacum, Nicotiana tabacum of the cultivar cv. Little Crittenden, rice plants, Lemna major or Lemna minor (duckweeds), safflower plants or any other plants that are both agriculturally propagated and amenable to genetic modification for the expression of recombinant or foreign proteins.

In one embodiment, the recombinant protein is expressed transiently, with or without P19, in N. benthamiana. In another embodiment, the nucleic acid molecule encoding the recombinant protein is integrated into the genome of an N. tabacum plant, which can be used thereafter for transgenic expression. In a preferred embodiment, the N. tabacum plant is of the cultivar cv. Little Crittenden (LCR) and the recombinant protein is co-expressed with P19. As described in Example 1, LCR was the only cultivar identified that does not induce hypersensitive response (HR) in the presence of P19. This tobacco cultivar can thus be utilized effectively in conjunction with P19-based transgenic expression systems.

In one embodiment, the recombinant protein and P19 are co-expressed in an RNAi-based glycomodified tobacco plant. In a preferred embodiment, the plant is an N. benthamiana plant. In a more preferred embodiment the N. benthamiana plant exhibits RNAi-induced gene-silencing of endogenous fucosyltransferase (FT) and xylosyltransferase (XT) genes. As shown in Example 1, P19 can safely be used with RNAi-based glycomodifed N. benthamiana expression hosts for the production of recombinant proteins such as antibodies without altering the glycan profile of the recombinant protein.

As used herein, the phrase “RNAi-based glycomodified tobacco plant” means a tobacco plant that expresses polypeptides with altered glycan profiles, wherein the altered profiles result from the use of interfering RNA (RNAi) gene-silencing technology. Plant-specific sugar residues on the N-glycan core, namely core α1,3-fucose and β1,2-xylose, are immunogenic in mammals (Bardor et al., 2003; Jin et al., 2008). Because of the existence of multiple endogenous FT and XT genes in most plants, modified glycosylation patterns are preferably created with the use of RNAi technology (Cox et al., 2006; Sourrouille et al., 2008; Strasser et al., 2008).

The phrase “growing a plant or plant cell to obtain a plant that expresses a recombinant protein” includes both growing transgenic plant cells into a mature plant as well as growing or culturing a mature plant that has received the nucleic acid molecules encoding the recombinant protein. One of skill in the art can readily determine the appropriate growth conditions in each case.

In a specific embodiment, expression vectors containing the recombinant nucleic acid molecules are introduced into A. tumefaciens strain by electroporation procedures. The N. benthamiana plants can be vacuum infiltrated according to the protocol described by Marillonnet et al. (2005) and Giritch et al. (2006) with several modifications. Briefly, all cultures can be grown at 28° C. and 220 rpm to a final optical density at 600 nm (OD₆₀₀) of 1.8. Equal volumes are combined and pelleted by centrifugation at 8,000 rpm for 4 minutes, resuspended and diluted by 10³ in infiltration buffer (10 mM 1-(N-morpholino)ethanesulphonic acid (MES) pH 5.5, 10 mM MgSO₄). Alternatively, each of the 5 Agrobacterium cultures could be grown to lower OD values and Beer's Law could be applied to determine the volumes of each culture required to make a bacterial suspension cocktail whereby the concentrations of each bacterial strain were equivalent. Alternatively, lower or higher concentrations of expression vectors could be used to optimize the expression of recombinant protein. Alternatively, higher or lower dilutions with infiltration buffer could be used.

The aerial parts of six-week-old N. benthamiana plants are submerged in a chamber containing the Agrobacterium tumefaciens resuspension solution, after which a vacuum (0.5 to 0.9 bar) is applied for 90 seconds followed by a slow release of the vacuum, after which plants were returned to the greenhouse for 8 days before being harvested. Alternatively, longer or shorter periods under vacuum, and/or vacuum release, could either/or/and be used. Alternatively, longer or shorter periods of growth in greenhouse could be used. Alternatively, standard horticultural improvement of growth, maximized for recombinant protein production could be used (see Colgan et al., 2010).

Alternately, instead of transient introduction of expression vectors containing the HC and LC coding sequences of an antibody, or the coding sequence of butyrylcholinesterase, stable transgenic plants could be made using one vector on which the nucleic acid molecule encoding the heavy chain variable region and the nucleic acid molecule encoding the light chain variable region may be introduced together in the same construct. In one embodiment, the nucleic acid molecule encoding the heavy chain variable region may be attached to the nucleic acid molecule encoding the light chain variable region by a linker in order to prepare a single chain variable region fragment (scFv).

In another embodiment, the nucleic acid molecule encoding the heavy chain and the nucleic acid molecule encoding the light chain may be introduced into the plant cell on separate expression vector nucleic acid constructs. In such an embodiment, the heavy chain and the light chain would be expressed from separate transgenic loci and then combine in the plant cell in order to prepare the antibody or antibody fragment.

Expression vector(s) containing antibody HC and LC genes would be introduced into Agrobacterium tumefaciens At542 or other suitable Agrobacterium isolates or other suitable bacterial species capable of introducing DNA to plants for transformation such as Rhizobium sp., Sinorhizobium meliloti, Mesorhizobium loti and other species (Broothaerts et al. 2005; Chung et al., 2006), by electroporation or other bacterial transformation procedures. Agrobacterium clones containing expression vectors would be propagated on Luria-Bertani (LB) plates containing rifampicin (30 mg/l) and kanamycin (50 mg/l), or other selectable media, depending on the nature of the selectable marker genes on the vector. Agrobacterium-mediated leaf disk transformation (Horsch et al. 1985; Gelvin, 2003), or similar protocols involving wounded tobacco (N. tabacum, variety 81V9 or tissue of other tobacco varieties such as are listed in Conley et al, 2009) or N. benthamiana or other plant species such as those of the Solanaceae, maize, safflower, Lemna spp., etc. would be infected with the Agrobacterium culture (OD600=0.6) and plated on Murashige and Skoog plus vitamins medium (MS; Sigma), supplemented with agar (5.8%; Sigma) and containing kanamycin (100 mg/l) or 500 cefotaxime (mg/L) or other selectable media, depending on the nature of the selectable marker genes on the expression vector, for selection of transformed plant cells. Production of shoots would be induced with naphthalene acetic acid (NAA; 0.1 mg/l; Sigma) and benzyl adenine (BA; 1 mg/l; Sigma) in the medium. For induction of roots, the newly formed shoots were moved to Magenta boxes (Sigma-Aldrich, Oakville, ON) on MS medium (as above) that was lacking NAA and BA. After roots are formed, plants would be transplanted to soil and could be raised in greenhouse culture. For plant transformation, as many as possible or at least 25 primary transgenic plants would be produced. ELISA and quantitative immunoblots would be performed on each plant to characterize levels of total and active antibody produced by the plants, respectively (Almquist et al., 2004; 2006; McLean et al., 2007; Olea-Popelka et al., 2005; Makvandi-Nejad et al., 2005).

After selection of antibody-expressing primary transgenic plants, or concurrent with selection of antibody expressing plants, derivation of homozygous stable transgenic plant lines would be performed. Primary transgenic plants would be grown to maturity, allowed to self-pollinate, and produce seed. Homozygosity would be verified by the observation of 100% resistance of seedlings on kanamycin plates (50 mg/L), or other selectable drug as indicated above. A homozygous line with single T-DNA insertions, that are shown by molecular analysis to produce most amounts of antibody, would be chosen for breeding to homozygosity and seed production, ensuring subsequent sources of seed for homogeneous production of antibody by the stable transgenic or genetically modified crop (Olea-Popelka et al., 2005; McLean et al., 2007; Yu et al., 2008).

Alternatively, the expression vector with both HC and LC genes, or 2 expression vectors (one with a HC gene and the other with a LC gene), could be used to transiently infect a plant or plant tissues, as described above, and tissue harvested as described above for subsequent purification of antibody.

The antibody or antibody fragment or the enzyme may be purified or isolated from the plants using techniques known in the art, including homogenization, clarification of homogenate and affinity purification. Homogenization is any process that crushes or breaks up plant tissues and cells and produces homogeneous liquids from plant tissues, such as using a blender, or juicer, or grinder, or pulverizer such as mortar and pestle, etc. Clarification involves either/and/or centrifugation, filtration, etc. Affinity purification uses Protein A or Protein G or Protein L or antibodies that bind antibodies; affinity purification for enzymes uses ligands that bind them, such as procainamide or huprine.

The following non-limiting examples are illustrative of the present invention:

### Example 1

### Experimental Procedures

### Plant Expression Vectors

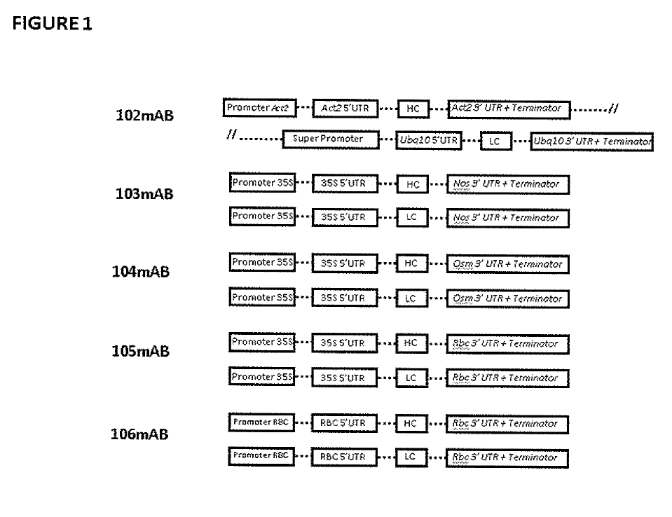

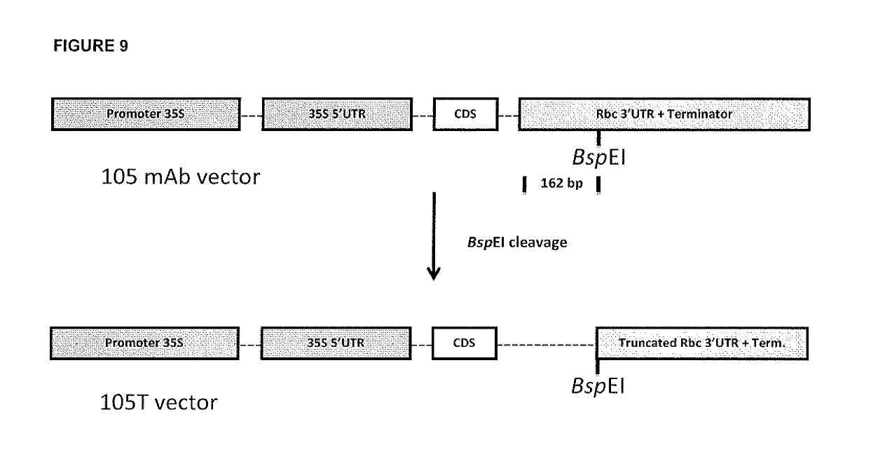

Four expression cassettes, namely 103-106, were synthesized and cloned into the T-DNA region of pICH14011, creating plasmids p103-p106 for producing recombinant proteins in Nicotiana species. The structures of the expression cassettes are depicted in FIG. 1. Expression cassettes 103-105 contain the 35S promoter and 5′UTR of the Cauliflower Mosaic Virus (CaMV; Genbank accession: AF140604), while 106 contains the promoter and 5′ UTR of ribulose bisphosphate carboxylase (rbc) small subunit gene from Chrysanthemum morifolium (Genbank accession: AY163904.1). Cassette 103 contains the 3′ UTR and terminator sequences from the nopaline synthase (nos) gene of Agrobacterium (Genbank accession: V00087.1), cassette 104 contains the 3′ UTR and terminator sequences from the osmotin (osm) gene of Oryza sativa (Genbank accession: L76377.1), and cassettes 105 and 106 carry the 3′ UTR and terminator sequences from the rbc gene of C. morifolium (Genbank accession: AY163904.1). The structures of another expression cassette is depicted in FIG. 9. This expression cassette was derived from p105 bp insertion of a coding sequence for a recombinant protein to be expressed in plants using the restriction endonuclease BspEI for ligation of the 3′ end of that coding sequence, resulting in a 162 bp deletion of the 5′ end of the Rbc 3′ UTR and terminator. The resulting plasmid is known as p105T.

The P19 protein from Tomato Bushy Stunt Virus (TBSV; Genbank accession: M21958) was cloned in cassette 103. The heavy and light chains of trastuzumab (Grohs et al., 2010) were cloned separately in expression cassettes 103-106. The heavy and light chains were both fused to the signal sequence from the basic chitinase gene of Arabidopsis thaliana (Genbank accession: AY054628) for secretion into the apoplast. All protein sequences were codon-optimized for expression in N. benthamiana. The codon-optimized nucleic acid molecule encoding P19 has the sequence shown in SEQ ID NO:1. The heavy and light chains of trastuzumab were also cloned in a single binary vector, designated as 102mAb, in which the heavy chain was driven by acting promoter from Arabidopsis thaliana (Genbank accession: NM_{—}112764) with A. thaliana acting UTRs and terminator region, and the light chain driven by the chimeric octopine and mannopine synthase promoter (Genbank accession: EU181146.1) with UTRs and terminator region of A. thaliana ubiquitin 10 (ubq10) gene (Genbank accession: L05361).

### Bacterial Transformation and Culture

Competent Agrobacterium tumefaciens A136 cells were transformed with the above-mentioned expression cassettes by a standard heat-shock method. Bacterial cultures were grown overnight at 28° C. in YEP medium (10 g Bacto peptone, 10 g yeast extract, and 5 g sodium chloride per liter, pH 7.0) supplemented with antibiotics. Unless otherwise stated, dense overnight cultures (up to OD₆₀₀=2.5) for each vector were adjusted to an OD₆₀₀ of 0.2 in Agroinfiltration buffer (AIB) containing 10 mM 2-(4-morpholino)-ethanesulfonic acid (MES), 10 mM MgSO4, pH 5.5, and mixed together to create an Agrobacterium infiltration cocktail (AIC) for plant treatment.

### Transient Expression Assay and Sample Preparation

Plants were grown in the greenhouse and fed high nitrate fertilizer (N:P:K=20:8:20) daily at 1 g/l (Plant Products, Brampton, Ontario, Canada), adjusted to pH 6.0 with 20% H₃PO₄. To transiently express trastuzumab, an AIC containing two Agrobacterium strains, each harboring one of the antibody chains, were used to infiltrate plant leaves. All plants were treated at the 4- to 6-week stage, either by spot or whole-plant infiltration. Shortly after treatment, the plants were placed back in the greenhouse for a certain period of time prior to harvest, depending on the expression vector. During this period, plants were only fed water. When harvesting wholly infiltrated plants, newly emerged leaves were discarded and the infiltrated leaves were separated from the stems and stored at −80° C. until further processing. For spot infiltrations, 100 mg of leaf tissue from the infiltrated area was weighed and stored at −80° C. until further processing.

Approximately 100 mg of leaf tissue was mixed with 300 μl of extraction buffer containing phosphate buffered saline (PBS: 8 g NaCl, 0.2 g KCl, 1.44 g Na₂HPO₄, and 0.24 g KH₂PO₄ per liter) and 10 mM EDTA, pH 6.8 (PBSE buffer). Samples were disrupted in a 2 nil microfuge tube containing two stainless steel ball bearings for 5 minutes using a TissueLyser (Qiagen) to prepare a crude protein extract, which was aliquoted into small volumes and stored at −20° C.

### Western Blotting

A frozen aliquot was thawed, and spun in a refrigerated bench-top centrifuge at >13,000 rpm for 1 minute to clarify the crude extract for protein quantitation. Either a Bradford or a BCA (Pierce) assay was used to determine protein concentration of the once-thawed crude extracts. Thirty μg of total soluble protein (TSP) per sample was loaded in each well on an 8% SDS-polyacrylamide gel. The separated proteins were blotted on a polyvinylidene fluoride (PVDF) membrane and probed for antibody presence with a mix of alkaline phosphatase conjugated anti-human γ and κ antibodies (Sigma Aldrich, Cat# A3312 and A3813), diluted to 1:10,000 in PBS (pH 7.4) using NBT/BCIP (Thermo Scientific, Cat#34042) as substrate. Blots were developed for 2-5 minutes, depending on the experiment.

### ELISA

Enzyme-linked immunosorbent assay (ELISA) was used to quantitate the amount of antibody present in the crude protein extract of treated plants. ELISA plates were coated overnight at 4° C. with a mouse polyclonal anti-human IgG1 (Sigma Aldrich, Cat#15885) capture antibody at 0.6 μg/ml in PBS. Human IgG1 standard (Athens Research and Technology, Cat#16-16-090707) spiked in 5 μg of untreated crude protein extract was used as a standard. The standard curve was generated using human IgG1, which allowed for antibody detection over a range spanning three orders of magnitude (0.1-100 ng/well). Crude extract from each treated sample was loaded on an ELISA plate as two-fold dilutions in triplicate. Final antibody concentration was calculated by averaging the mean antibody concentration for three crude extract dilutions. A second anti-human antibody conjugated to HRP from rabbit (Abcam, Cat # ab6759) was used for detection, using TMB-ELISA (Thermo Scientific, Cat#34022) as substrate. The plates were allowed to develop for 15 minutes before the reaction was stopped with 0.5M H₂SO₄.

### Antibody Purification

Antibodies were purified essentially as described by Grohs et al. (2010).

### N-glycosylation Analyses

N-glycan analyses of purified mAbs were carried out by liquid-chromatography electrospray ionization-mass spectrometry (LC-ESI-MS) of tryptic glycopeptides as recently described (Stadlmann et al., 2008). Briefly, the purified samples were submitted to reducing SDS PAGE and the 55 kD band corresponding to the HO was cut from the gel, S-alkylated, digested with trypsin, eluted from the gel fragment with 50% acetonitril and separated on a Biobasic C18 column (150×0.32 mm, Thermo Electron) with a gradient of 1%-80% acetonitrile containing 65 mM ammonium formate pH 3.0. Positive ions were detected with a Q TOF Ultima Global mass spectrometer (Waters, Milford, Mass., USA). Summed and deconvoluted spectra of the glycopeptides elution range were used for identification of glycoforms. This method generates two glycopeptides that differ by 482 Da (glycopeptide 1, EEQYNSTYR; glycopeptide 2 TKPREEQYNSTYR).

### Results

### The Untranslated Regions Used in Recombinant Expression of Trastuzumab Significantly Impact Antibody Accumulation

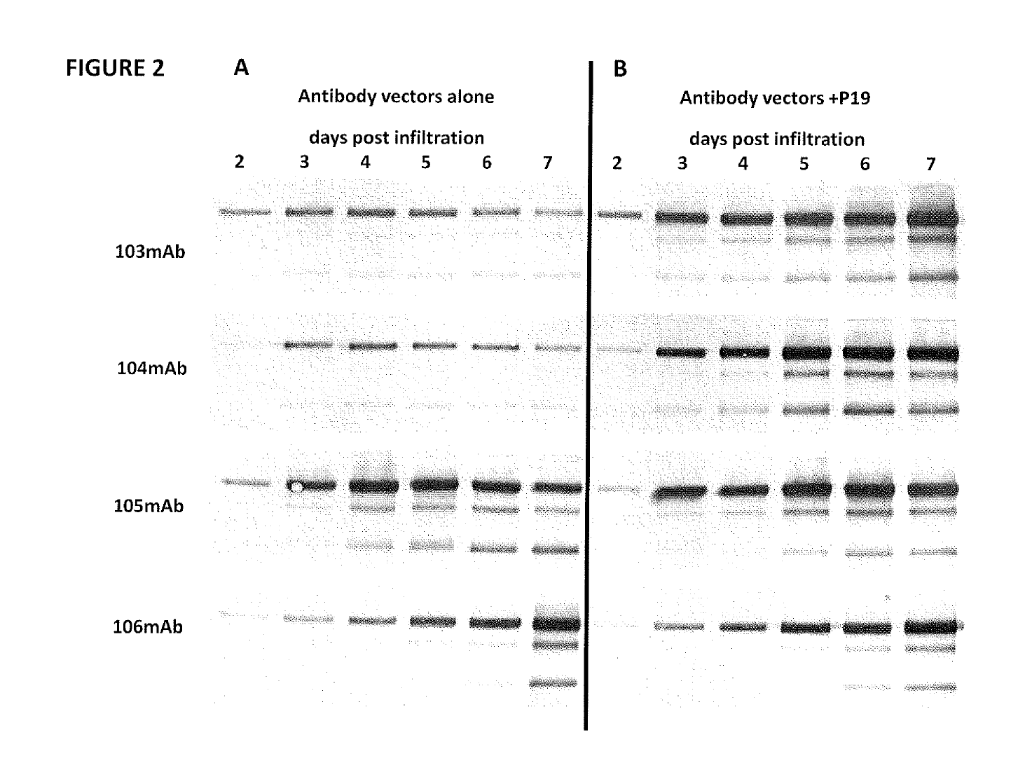

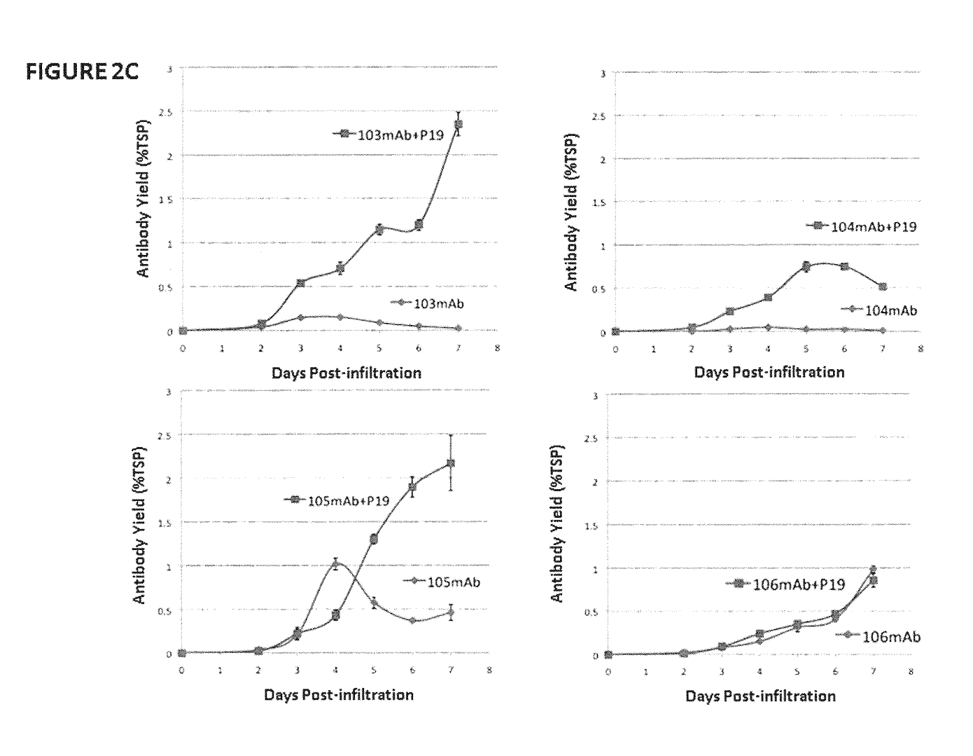

Trastuzumab is a therapeutic antibody used in the treatment of HER2+ breast cancer (Baselga et al., 1998; Lewis et al., 1993). To produce this antibody in Nicotiana hosts, its heavy (HC) and light (LC) chains were cloned into plant expression cassettes and placed either on a single binary vector (102mAb), or on separate binary vectors (103-106HC and 103-106LC), in which case they were co-expressed (referred to as vector sets 103mAb-106mAb) to produce the fully assembled antibody. The different expression cassettes were designed to carry different combinations of promoters, 5′UTRs, and 3′ UTR/terminators (FIG. 1). Trastuzumab was transiently expressed in N. benthamiana using vector sets 103mAb-106mAb to compare the levels of recombinant antibody production. A 7-day expression time-course with whole-plant vacuum infiltration showed a considerable difference in the dynamics and maximal antibody expression among the four vector sets (FIG. 2A). Vectors 105mAb and 106mAb resulted in higher maximal antibody accumulation compared to 103mAb and 104mAb. Antibody expression peaked at 3-4 days post-infection (d.p.i.) for 103mAb and 104mAb, and at 4-5 d.p.i. for 105mAb, whereas 106mAb showed a steady increase in expression up to 7 days post-infection. Maximal antibody levels were achieved with the 105mAb and 106mAb vector sets, estimated by ELISA at ≈1% of TSP. The protein content of tobacco leaves has been estimated at approximately 2% of the fresh weight (Stevens et al., 2000), therefore antibody expression at 1% of TSP translates into ≈200 mg of antibody per kg of fresh weight (FW). A 20-fold range in antibody accumulation was observed when the different vectors were compared. Therefore, different UTR elements fused to the codes for the heavy and light chains of trastuzumab significantly affect the accumulation of the fully assembled antibody when transiently expressed. In addition, N-glycosylation profiles of mAbs were determined by LC-ESI-MS (liquid-chromatography electrospray ionization-mass spectrometry). Typically mAbs exhibited a largely homogeneous GnGnXF³ oligosaccharide pattern with plant specific β1,2-xylose and core α1,3-fucose residues (see last section of Results).

P19 does not have a Similar Boosting Effect on the Expression of Trastuzumab from Different Expression Vectors

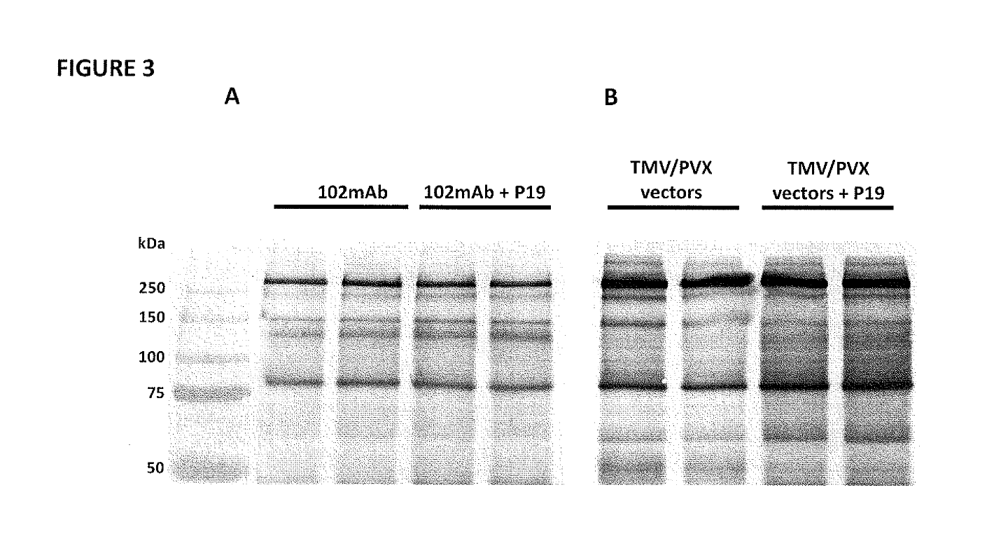

The effect of co-expressing P19 with vectors 103mAb-106mAb was analyzed over 7 days in N. benthamiana (FIGS. 2B and 2C) to determine whether the different UTR combinations had any effect on the ability of P19 to boost antibody expression, as previously reported (Saxena et al., 2011; Vezina et al., 2009). All Agrobacterium cultures used in this experiment were adjusted to an OD₆₀₀ of 0.2. Not all vector sets were positively affected by P19 (FIG. 2C, Table 1). For 103mAb and 104mAb, P19 resulted in a 15-fold increase in the concentration of trastuzumab, although maximal expression was almost 3-fold greater with 103mAb compared to 104mAb, both without and with P19. For 105mAb, P19 only resulted in a ≈2-fold increase in the concentration of trastuzumab, from 1% to approximately 2.1% of TSP. Antibody accumulation with vector set 106mAb, which contained only plant-derived UTRs, was unaffected by P19. Antibody expression peaked for 103mAb and 105mAb when co-expressed with P19 at just over 2% of TSP. It was also noted that P19 changed the peak time of expression for the vectors 103mAb, 104mAb, and 105mAb (FIG. 2C). The ability of P19 to boost expression of trastuzumab with vector 102mAb was also tested. 102mAB is similar to 106mAb, i.e., it only contains plant-derived UTRs (FIG. 1). Similar to 106mAb, P19 did not affect the antibody expression level of 102mAb (FIG. 3A). P19 was also found to have no boosting effect on trastuzumab expressed with Tobacco Mosaic Virus-(TMV) and Potato X Virus-based (PVX) deconstructed vectors (Grohs et al., 2010) (FIG. 3B).

### Boosting Effect of P19 on Recombinant Antibody Expression is Concentration Dependent

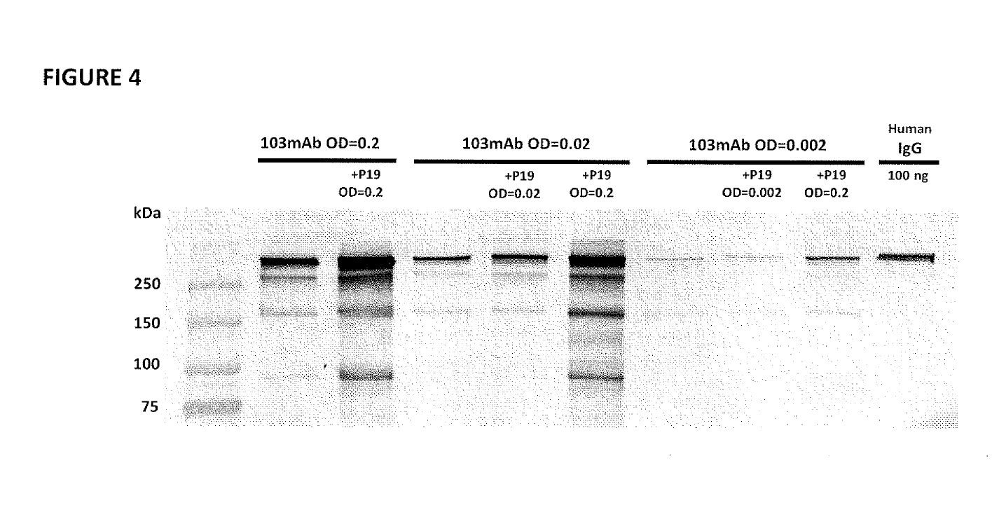

It is believed that in order for P19 to exert its biological function for a successful TBSV infection, it has to accumulate beyond a certain threshold concentration in plant cells (Qiu et al., 2002; Scholthof et al., 1999). When expressed transiently, co-expression of P19 with 103mAb at an Agrobacterium concentration of OD₆₀₀=0.2 significantly boosted antibody production. Since the expression level of recombinant proteins are, for the most part, lower for a transgenic versus transient expression, co-infiltration of 103mAb with P19 was examined at lower Agrobacterium concentrations to simulate transgenic-like levels of P19 accumulation in N. benthamiana. Infiltration of 103mAb vectors alone at OD₆₀₀ values of 0.2, 0.02, and 0.002 resulted in different levels of antibody production, with a direct correlation between the applied Agrobacterium concentration and antibody expression level (FIG. 4). The ability of P19 to boost antibody expression with 103mAb progressively diminished when P19 was applied at lower concentrations, while co-expressing P19 at OD₆₀₀=0.2 resulted in a significant increase in antibody expression irrespective of 103mAb concentration (FIG. 4). Thus, unless P19 can be expressed at very high levels, its utility in transgenic production of recombinant proteins may be limited due to its concentration-dependent mode of action.

### Tobacco Varieties Respond Differently to P19

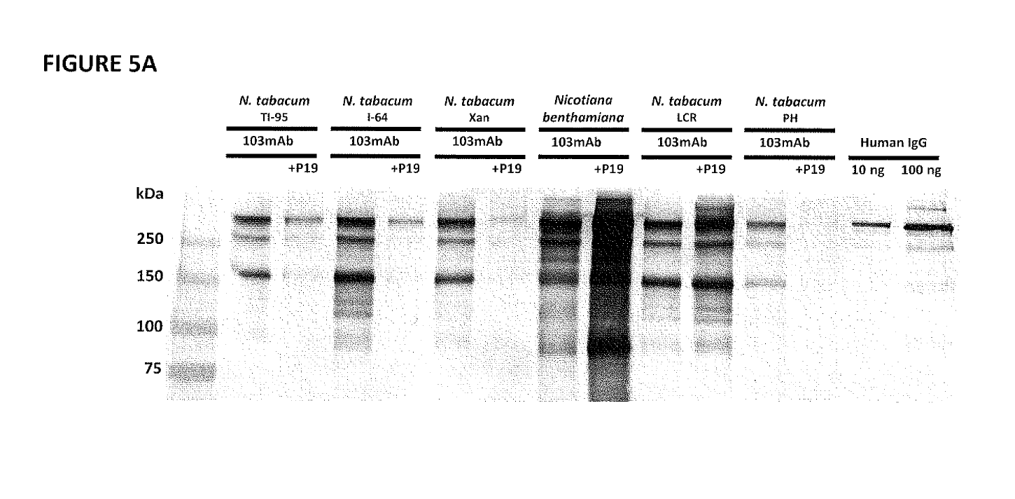

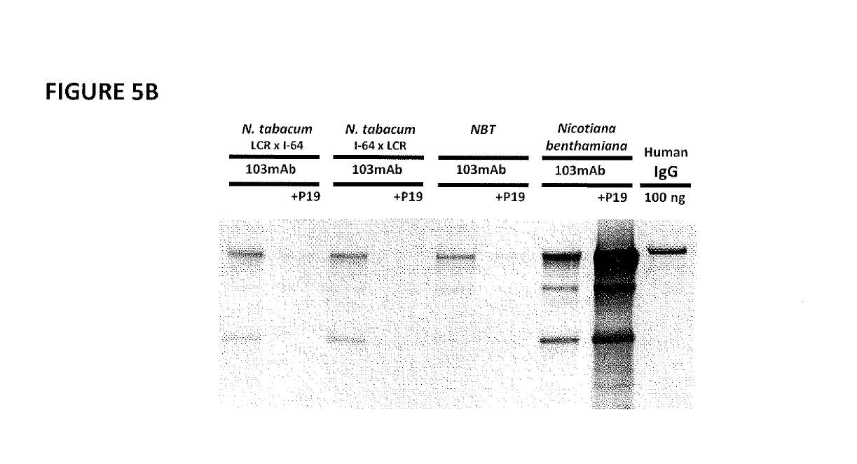

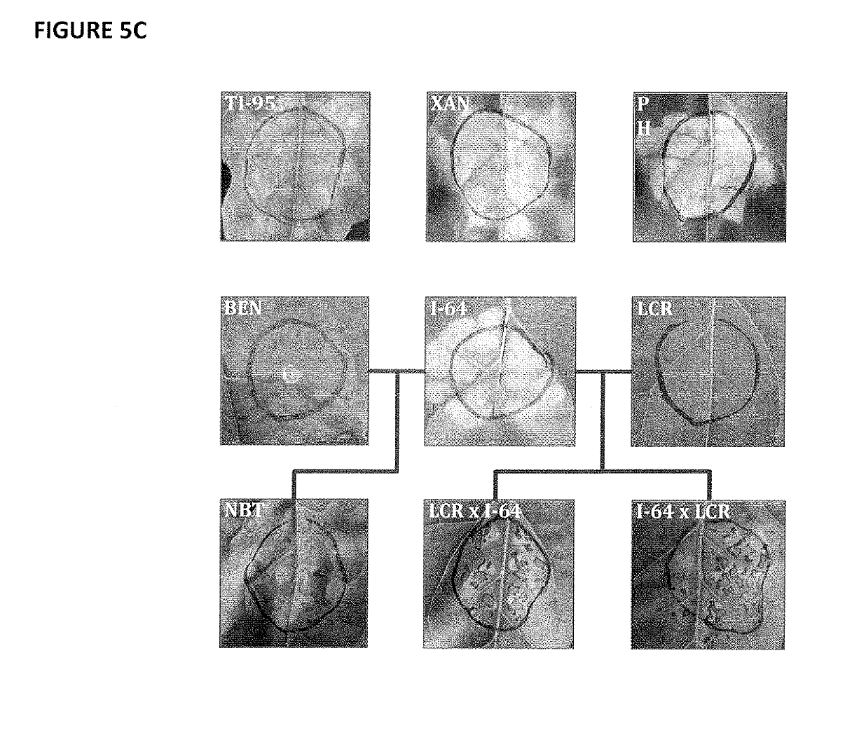

To utilize P19 for transgenic production of recombinant proteins in plants, it is imperative that P19 does not adversely affect plant growth, and more importantly, gene expression. As discussed, when expressed transiently at high levels in N. benthamiana, P19 effectively boosts recombinant protein levels. However, due to having a much greater biomass, N. tabacum is often selected over N. benthamiana for transgenic production. The downside of using N. tabacum is the development of necroses at the site of infection by 7 days after the introduction of recombinant P19 to leaf cells via Agroinfiltration (Angel et al., 2011). The reaction that is triggered by P19 in N. tabacum, also known as the hypersensitive response, has been well documented but only tested in two tobacco cultivars (Angel et al., 2011; Jovel et al., 2011; Siddiqui et al., 2008). To identify a tobacco that could be used with P19 in a transgenic expression system, five cultivars were screened for the development of the hypersensitive response. Trastuzumab was transiently expressed with 103mAb, alone or together with P19 by spot infiltration of 5-week-old plants. Four out of five tobacco cultivars, namely I-64, TI-95, Xanthi, and Petite Havana H4, showed a marked decrease in antibody expression at day 6 when co-expressed with P19, compared to antibody expression without P19 (FIG. 5A). Conversely, the co-expression of P19 with 103mAb in N. tabacum cv. Little Crittenden (LCR) resulted in a significant boost in trastuzumab expression (FIG. 5B). Furthermore, the tobacco cultivars that showed a decrease in antibody expression in the presence of P19 also showed a marked discoloration of the treated areas after 3 days, while the infiltrated areas of N. benthamiana and N. tabacum cv. LCR were unaffected up to 10 days post-infection (shown in FIG. 5C at 5 d.p.i). These results indicate that N. tabacum cv. Little Crittenden can be used as a host for transgenic production of recombinant proteins using P19.

Reciprocal crosses were made between tobacco cultivars 1-64 and LCR to look at the manner in which induction of the hypersensitive response by P19 is inherited in N. tabacum. The induction of this response in a sterile cross between N. benthamiana and N. tabacum, named NBT, was also tested. Five-week-old seedlings were infiltrated with 103mAb, with or without P19. The level of trastuzumab was reduced in all the crosses at 5 d.p.i. when 103mAb was co-expressed with P19 (FIG. 5B). This reduction in antibody expression correlated with discoloration of the treated leaves, followed by necrosis (FIG. 5C). These results indicate that the putative R gene responsible for triggering the HR is nuclear, and that LCR is homozygous recessive for that gene.

### P19 Does not Affect the Silencing of Fucosyltransferase and Xylosyltransferase Activity in RNAi-Based Glycomodified Nicotiana Benthamiana

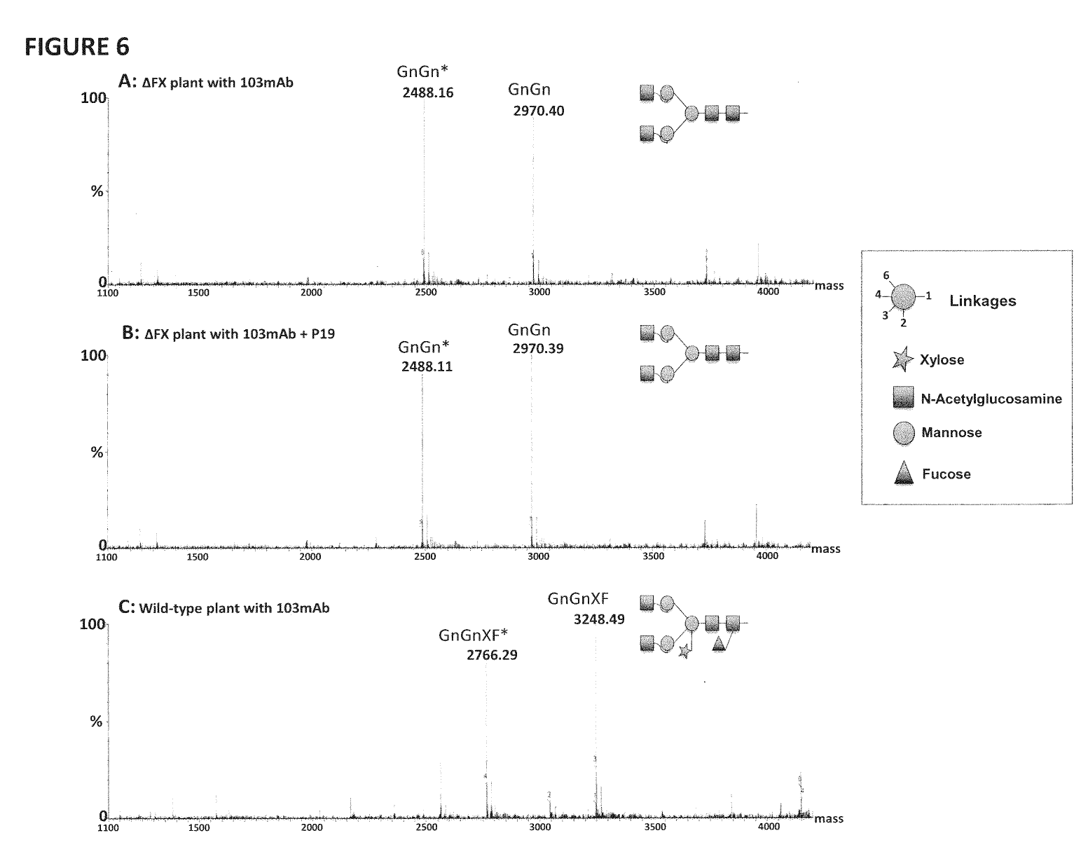

RNAi based silencing is a commonly used method to modify the N-glycosylation pattern towards human like structures in plants (Cox et al., 2006; Sourrouille et al., 2008; Strasser et al., 2008). This strategy was also applied to eliminate plant specific N-glycan residues (i.e. xylose and core α1,3 fucoce) in Nicotiana benthamiana (ΔXTFT) by the down-regulation of the respective enzymes fucosyltransferase and xylosyltransferase (Cox et al., 2006; Sourrouille et al., 2008; Strasser et al., 2008). The possible adverse effects of P19 in such plants had yet to be determined. Thus, ΔXTFT mutants were vacuum infiltrated with the mAb103 vectors with or without P19. Trastuzumab was purified 5 days post-infiltration and analyzed by LC-ESI-MS to determine the N-glycosylation pattern (FIG. 6 B, C). Interestingly, both 103mAb versions carried an identical N-glycosylation profile, lacking plant specific oligosaccharides. This suggests that P19 does not affect the RNAi silencing pathway responsible for the silencing of FT and XT in ΔXTFT while it interferes with the gene-silencing pathway that reduces transgene expression. These results indicate that P19 can be used for boosting recombinant protein levels expressed in an RNAi-based expression host without altering the protein's glycan profile.

### Transient Co-Infiltration of P19 Expression Vector Greatly Enhances the Expression of Two More Therapeutic Antibodies in Nicotiana Benthamiana.

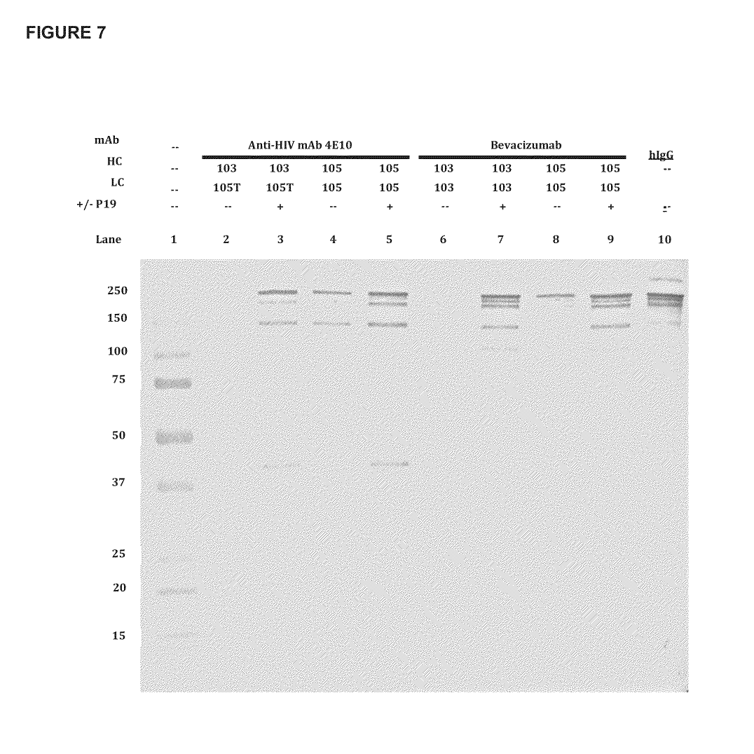

To demonstrate that P19 inhances the expression of other antibodies, coding sequences for the light and heavy chains of both anti-HIV mAb 4E10 and bevacizumab were synthesized for expression in plants (as above). Anti-HIV mAb 4E10 was first described in Buchacher et al. (1994) with its light chain sequence being available in the GenBank (http://www.ncbi.nlm.nih.gov/) as entry GI:122920218 and its heavy chain as a F_{d}/V_{H} in entry GI: 61680025. This antibody may be used as an anti-HIV vaccine or as a diagnostic reagent. The light and heavy chain sequences of bevacizumab are available from Drugbank (http://www.drugbank.ca/) as entry DB00112. Bevacizumab is used with standard chemotherapy for metastatic colon cancer; it also been approved for use in certain lung cancers, renal cancers, and glioblastoma multiforme of the brain. The heavy chain coding sequence for mAb 4E10, and both the light and heavy chain coding sequences for bevacizumab were cloned downstream of the Arabidopsis basic chitinase signal peptide (as above) and inserted into the 103 and 105 vectors. The light chain coding sequence for mAb 4E10 was also cloned downstream of the Arabidopsis basic chitinase signal peptide and inserted into the 105 vector and into a version of the 105 vector in which a 162 base-pair deletion of the 5′ end of the C. morifolium rbc gene terminator sequence was caused by cleavage at a BspEI site; this latter vector is referred to as p105T (i.e., 105-truncated). All eight vectors were introduced into Agrobacterium tumefaciens strain At542 (as above), then spot-infiltrated, with and without P19 (as above), into N. benthamiana leaf tissue in the combinations indicated in FIG. 7 and harvested after 7 days. Each Agrobacterium strain was applied at a final OD₆₀₀ of 0.2. Tissue harvests for each treatment included 3 or 4 spots, which were pooled and total soluble protein (TSP) was extracted as described in Garabagi et al. (2012a,b). A 10% non-reducing SDS-PAGE gel was run that included 10 mg TSP for each plant sample. Electrophoretically separated proteins were subsequently electrotransferred to PVDF membrane and probed with combined anti-γ and anti-K antibody probes conjugated to alkaline phosphatase (also described in Garabagi et al., 2012a). Results indicate that co-expression of P19 greatly enhances expression of both antibodies irrespective of antibody expression vector. In FIG. 7, the gel loading scheme is tabulated above the immunoblot image, and the size of each molecular weight marker band in lane 1 is given on the left in kDa; the human serum immunoglobulin quantification standard in lane 10 is 500 ng; mAb=monoclonal antibody; HC=heavy chain vector; LC=light chain vector.

### Transient Expression of a Therapeutic Enzyme, Human Butyrylcholinesterase (BChE) is Greatly Enhanced by Co-Expression of P19.

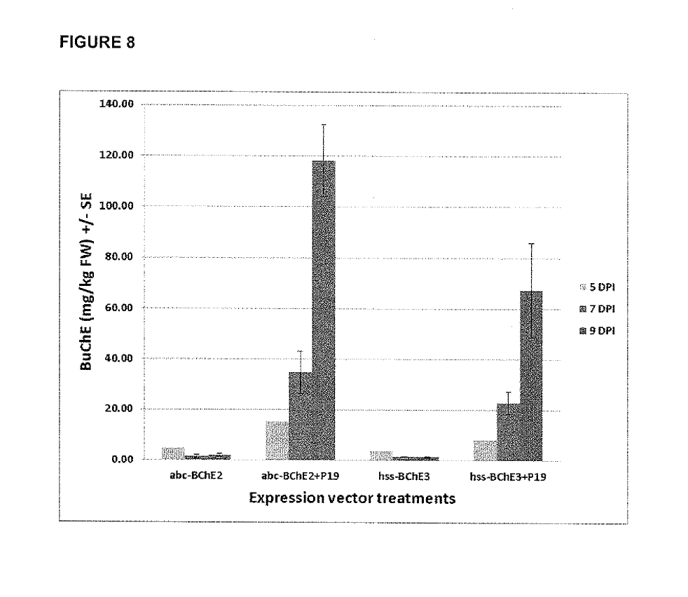

Butyrylcholinesterase ⁱs a non-specific cholinesterase enzyme that hydrolyses many different choline esters. In humans, it is found primarily in the liver and is encoded by the BCHE gene. It is being developed as an antidote to nerve-gas poisoning. Two different BChE expression vectors were constructed in p105T (described above): (1) with the Arabidopsis basic chitinase (abc) signal sequence and a synthetic BChE coding sequence optimized for expression in plants referred to as E2, i.e., abc-BChE2; and (2) with the native human BChE signal sequence (hSS) and another synthetic BChE coding sequence optimized for expression in plants referred to as E3, i.e., hSS-BChE3. These were introduced into whole N. benthamiana plants by vacuum infiltration (according to Garabagi et al., 2012a,b), either with or without the P19 (as above). Samples were taken at 5, 7 and 9 days post-infiltration (DPI) and BChE activity was measured by Ellman Assay (Ellman et al., 1961). Results of this experiment are presented in FIG. 8 where histogram bars present amounts of BChE in mg BChE/kg leaf tissue, by converting activity measurements to mass of enzyme using the specific activity conversion factor of 718.3 activity units per mg of BChE determined in Weber et al. (2010). Note that background readings (as determined with untreated plant extracts) have been subtracted for the presentation of these data. Histogram bars in FIG. 8 indicate the mean BChE activity measured in 2 samples from 2 plants (4 total repeats each) with associated standard error bars. These data indicate that co-expression of P19 greatly enhances expression of BChE.

### Discussion

In this example, three important issues are addressed regarding the application of P19 to improve recombinant protein production in plants: P19's ability to sufficiently boost recombinant protein expression; its potential adverse physiological effects in the expression host; and interference with the state of gene-silencing in RNAi-based glycomodified plants. The results described herein indicate that all three requirements are met and that P19 can be effectively utilized for transient expression of recombinant glycoproteins in RNAi-based expression hosts.

The highest antibody expression reports using suppressors of gene silencing to date have been on transient expression of mAb 2G12 with P19 at 400 mg/kg FW (Saxena et al., 2011) and mAb C5-1 with HcPro at 757 mg/kg FW (Vezina et al., 2009). Both of these mAbs were retained in the ER by the addition of auxiliary C-terminal tags. In the context of biosimilar therapeutic antibodies, however, ER retention signals are problematic since they add extra amino acids to the primary sequence of the innovator protein. In addition, ER typical oligomannisidic N-glycsoylation is for most therapeutic proteins untypical and thus unwanted.

P19 is herein shown to enhance expression of the model therapeutic mAb trastuzumab that is targeted to the apoplast using classical binary vectors at about 2.3% of TSP, or ≈460 mg/kg FW. Furthermore, regulatory genetic elements used in recombinant constructs determine whether or not P19 can boost expression. This was demonstrated by co-expressing P19 together with the heavy and light chains of trastuzumab cloned in five different expression vectors containing different 5′ and 3′ UTRs. The results described herein indicate that transcripts with at least one virus-derived UTR, such as in 103mAb, 104mAb, and 105mAb, were boosted by P19. This suggests that the transcripts of these expression cassettes are subjected to RNAi silencing, albeit to different extents, and therefore are boosted in the presence of a suppressor of a gene silencing. In contrast, transcripts that only contained plant derived UTRs, such as 106mAb and 102mAb, were unaffected by P19, suggesting they were not subjected to any significant RNAi silencing during the observation period. These findings may have direct implications in the design of expression vectors.

P19 is herein also shown to enhance expression of another therapeutic mAb, namely bevacizumab, using two of the vectors described in this application. P19 is herein also shown to enhance expression of another therapeutic mAb, namely anti-HIV mAb 4E10, using three of the vectors described in this application. These three examples illustrate the potential for P19 to enhance the expression of most any antibody using the vector system presented in this application.

P19 is herein also shown to enhance expression of a potential therapeutic enzyme, namely butyrylcholinesterase, using one of the vectors described in this application with either the Arabidopsis basic chitinase signal sequence or with the native human butyrylcholinesterase signal sequence, and using either of two synthetic coding sequences optimized for plant expression of the same identical butyrylcholinesterase polypeptide. This example illustrates the potential for P19 to enhance the expression of therapeutic proteins other than antibodies, such as enzymes.

It is apparent that the vectors described in this application can produce different therapeutic proteins, and that expression of proteins from these vectors in planta can be enhanced by co-expression of the P19 gene.

It is also apparent from several reports on constitutive transgenic expression of P19 that the onset of adverse effects occurs only when the protein is used in high titers, especially since high-level constitutive expression of P19 is known to be lethal in Arabidopsis (Dunoyer et al., 2004). This dose-dependent functionality of P19 is supported by the fact that transgenic tobacco cv. Xanthi is tolerant to P19 at low levels (Siddiqui et al., 2008), while the protein generates necrosis in tobacco cvs. Samsun and NC95 leaves when transiently expressed at high titers (Angel et al., 2011). Inducible expression systems that are capable of producing high levels of recombinant protein have been employed to circumvent the lethality that is caused by producing high titers of P19 in transgenic systems. Nonetheless, unfavorable effects such as malformed leaves and flowers appear when P19 is expressed with such systems at high levels, such as with the pOp/LhG4 transactivation systems described by Stay et al. (2009). Since transgenes generally express higher in transient as opposed to transgenic settings (Garabagi et al. 2012, in press), the amount of P19 protein produced by transient infiltration at concentrations of OD₆₀₀=0.02 and lower was shown not to significantly enhance trastuzumab levels. On the other hand, higher P19 concentrations (OD₆₀₀=0.2) caused a 15-fold increase in antibody levels (FIG. 4). These results support the idea of dose-dependent functionality of P19 in the context of boosting recombinant protein expression. However, the titer at which P19 exerts its boosting effect causes a hypersensitive response in most tobacco cultivars (FIG. 5A).

Recent reports on the induction of the hypersensitive response in N. tabacum cvs. Samsun and NC95 describe a physiological response that involves local induction of RNAi silencing (Jovel et al., 2011) followed by necrosis (Angel et al., 2011). This response is likely triggered by the product of a putative R gene (Angel et al., 2011). As described in this example, a tobacco cultivar, Little Crittenden, was identified which did not trigger a hypersensitive response when exposed to high titers of P19 (FIGS. 5A and 5C). All other tested tobacco cultivars showed a marked decrease in antibody production in the presence of P19 compared to the expression of the antibody vectors alone, indicating the induction of an RNAi silencing mechanism that overrides the suppression of gene-silencing by P19. These findings are in line with recent reports on the induction of the hypersensitive response in N. tabacum that includes a local induction of RNAi silencing (Angel et al., 2011; Jovel et al., 2011). The results described herein also indicate a dominant mode of inheritance for this trait, also in accordance with previously described characteristics of R genes (Moffett, 2009), and that the LCR cultivar has a recessive mutation in this gene. Thus, this tobacco genotype and others that have similar R gene mutations may lend themselves to transgenic expression of recombinant proteins using P19 when used with a system capable of generating high titers of the protein. LCR can be used as a model for determining the number of genes involved in the hypersensitive response to P19.

Despite the ability of plants to carry out complex glycosylation, a possible bottleneck for their use as a versatile expression platform for therapeutic antibodies is the presence of plant specific N-glycan residues, i.e. xylose and core α1,3-fucose. Such non-mammalian oligosaccharides might change the biological activity of a given protein or might even induce unwanted adverse side effects upon therapeutic application. As expected, during the experiments described herein, plant-typical N-glycosylation was detected on trastuzumab. To circumvent this problem RNAi technology had been used to generate a plant line that lacks unwanted plant specific N-glycan residues (Strasser et al., 2008). Monoclonal antibodies produced in this mutant (ΔXTFT) carry complex human-like N-glycans lacking plant-specific glycosylation. Moreover, mAbs with such a glycoengineered profile have also shown increased effector functions compared to their mammalian cell-derived counterparts (Forthal et al., 2010; Zeitlin et al., 2011). Thus, such glycosylation mutant plants may serve as valuable expression platforms for the generation of therapeutic mAbs. However, whether the use of P19 would perturb the silencing of XT and FT in ΔXTFT mutants has not been investigated yet. Based on previous reports on the cellular targets of P19, including a documented case of interference with the siRNA silencing pathway in a transgenic plant line (Ahn et al., 2011), P19 was expected to interfere with the RNAi-induced gene-silencing of XT and FT in glycomodified N. benthamiana. Surprisingly, mAbs expressed in ΔXTFT with and without P19 were in both cases completely devoid of xylose and fucose residues. The virtually identical glycan profiles of the two antibodies indicate that the silencing of FT and XT is unaffected by P19 (FIG. 6). Thus, it appears that P19 can be used effectively in combination with RNAi-based glycomodified hosts for the production of therapeutic glycoproteins in both transient and stable expression systems.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Maximal trastuzumab level produced with different expression vectors.

| Vector | Max Expression | Max Expression | Expression |
|---|---|---|---|
| Set | (% TSP) | with P19 (% TSP) | increase with P19 |
| 103mAb | 0.15 ± 0.01 | 2.35 ± 0.13 | 15.6x |
| 104mAb | 0.05 ± 0.01 | 0.75 ± 0.04 | 15.0x |
| 105mAb | 1.02 ± 0.07 | 2.17 ± 0.31 | 2.1x |
| 106mAb | 0.98 ± 0.04 | 0.85 ± 0.08 | 1.0x |

SEQUENCE LISTING
SEQ ID NO: 1 P19 nucleotide sequence codon-
optimized for expression in Nicotiana.
ATGGAAAGGGCTATTCAGGGAAATGATGCTAGAGAGCAGGCTAATTCTGA
AAGATGGGATGGTGGATCTGGTGGAACTACTTCTCCATTCAAGCTTCCAG
ATGAGTCTCCATCTTGGACTGAGTGGAGGCTTCATAACGATGAGACTAAC
TCCAATCAGGATAACCCACTCGGATTCAAAGAATCTTGGGGATTCGGAAA
GGTTGTGTTCAAGCGTTACCTTAGGTATGATAGGACTGAGGCTTCACTTC
ATAGGGTTCTCGGATCTTGGACTGGTGATTCTGTTAACTACGCTGCTTCT
CGTTTTTTTGGATTCGATCAGATCGGATGCACTTACTCTATTAGGTTCAG
GGGAGTGTCTATTACTGTTTCTGGTGGATCTAGGACTCTTCAACACCTTT
GCGAGATGGCTATTAGGTCTAAGCAAGAGCTTCTTCAGCTTGCTCCAATT
GAGGTTGAGTCTAACGTTTCAAGAGGATGTCCAGAAGGTACTGAGACTTT
CGAGAAAGAATCCGAGTGA

### FULL CITATIONS FOR REFERENCES REFERRED TO IN THE SPECIFICATION

Ahn J W, Lee J S, Davarpanah S J, Jeon J H, Park Y I, Liu J R and Jeong W J (2011) Host-dependent suppression of RNA silencing mediated by the viral suppressor p19 in potato. Planta.
Almquist K. C., Niu Y., McLean M. D., Mena F. L., Yau K. Y., Brown K., Brandle J. E. and Hall J. C. (2004) Immunomodulation confers herbicide resistance in plants. Plant Biotechnol J 2, 189-97.
Angel C A, Hsieh Y C and Schoelz J E (2011) Comparative analysis of the capacity of tombusvirus P22 and P19 proteins to function as avirulence determinants in Nicotiana species. Mol Plant Microbe Interact 24:91-99.
Bardor M, Faveeuw C, Fitchette A C, Gilbert D, Galas L, Trottein F, Faye L and Lerouge P (2003) Immunoreactivity in mammals of two typical plant glycoepitopes, core alpha(1,3)-fucose and core xylose. Glycobiology 13:427-434.
Baselga J, Norton L, Albanell J, Kim Y M and Mendelsohn J (1998) Recombinant humanized anti-HER2 antibody (Herceptin) enhances the antitumor activity of paclitaxel and doxorubicin against HER2/neu overexpressing human breast cancer xenografts. Cancer Res 58:2825-2831.
Baulcombe D (2004) RNA silencing in plants. Nature 431:356-363.
Brodersen P and Voinnet O (2006) The diversity of RNA silencing pathways in plants. Trends Genet 22:268-280.
Brodsky, L. I.; Ivanov, V. V.; Kalaidzidis, Y. L.; Leontovich, A. M.; Nikolaev, V. K.; Feranchuk, S. I.; Drachev, V. A. GeneBee-NET: internet-based server for analyzing biopolymers structures. Biochemestry 1995, 60, 923-928.
Broothaerts W, Mitchell H J, Weir B, Kaines S, Smith L M A, Yang W, Mayer J E, Roa-Rodriguez C, Jefferson R A (2005) Gene transfer to plants by diverse species of bacteria, Nature 433:629-633.
Buchacher, A., R. Predl, K. Strutzenberger, W. Steinfellner, A. Trkola, M. Purtscher, G. Gruber, C. Tauer, F. Steindl, A. Jungbauer, and H. Katinger. 1994. Generation of human monoclonal antibodies against HIV-1 proteins; electrofusion and Epstein-Barr virus transformation for peripheral blood lymphocyte immortalization. AIDS Research and Human Retroviruses 10:359-69.
Burgyan J, Lakatos L, Szittya G and Silhavy D (2004) Molecular mechanism of RNA silencing suppression mediated by p19 protein of tombusviruses. Embo Journal 23:876-884.
Carthew R W and Sontheimer E J (2009) Origins and Mechanisms of miRNAs and siRNAs. Cell 136:642-655.
Chung S. M., Vaidya M. and Tzfira T. (2006) Agrobacterium is not alone: gene transfer to plants by viruses and other bacteria. Trends Plant Sci 11, 1-4.
Colgan R., Atkinson C. J., Paul M., Hassan S., Drake P. M., Sexton A. L., Santa-Cruz S., James D., Hamp K., Gutteridge C. and Ma J. K. Optimisation of contained Nicotiana tabacum cultivation for the production of recombinant protein pharmaceuticals. Transgenic Res 19, 241-56.
Cox K M, Sterling J D, Regan J T, Gasdaska J R, Frantz K K, Peele C G, Black A, Passmore D, Moldovan-Loomis C, Srinivasan M, Cuison S, Cardarelli P M and Dickey L F (2006) Glycan optimization of a human monoclonal antibody in the aquatic plant Lemna minor. Nature biotechnology 24:1591-1597.
Dunoyer P, Lecellier C H, Parizotto E A, Himber C and Voinnet O (2004) Probing the microRNA and small interfering RNA pathways with virus-encoded suppressors of RNA silencing. Plant Cell 16:1235-1250.
Ellman, G. L., Courtney, K. D., Andres, V., Jr., Featherstone, R. M., A new and rapid colorimetric determination of acetylcholinesterase activity. Biochem. Pharmacol. 7: 88-95, 1961.
Forthal D N, Gach J S, Landucci G, Jez J, Strasser R, Kunert R and Steinkellner H (2010) Fc-glycosylation influences Fcgamma receptor binding and cell-mediated anti-HIV activity of monoclonal antibody 2G12. Journal of immunology 185:6876-6882.
Gardiner-Garden, M.; Frommer, M. CpG islands in vertebrate genomes. J. Mol. Biol. 1987, 196, 261-282.
Garabagi, F., E. Gilbert, A. Loos, M. D. McLean, and J. C. Hall. 2012a. Utility of the P19 suppressor of gene-silencing protein for production of therapeutic antibodies in Nicotiana expression hosts. Plant Biotechnol J 10:1118-28.
Garabagi, F., M. D. McLean, and J. C. Hall. 2012b. Transient and stable expression of antibodies in Nicotiana species. Methods Mol Biol 907:389-408.
Gelvin S. B. (2003) Agrobacterium-mediated plant transformation: the biology behind the “gene-jockeying” tool. Microbial Mol Biol Rev 67, 16-37.
Giritch A., Marillonnet S., Engler C., van Eldik G., Botterman J., Klimyuk V. and Gleba Y. (2006) Rapid high-yield expression of full-size IgG antibodies in plants coinfected with noncompeting viral vectors. Proc Natl Acad Sci USA 103, 14701-6.
Gomord V, Fitchette A C, Menu-Bouaouiche L, Saint-Jore-Dupas C, Plasson C, Michaud D and Faye L (2010) Plant-specific glycosylation patterns in the context of therapeutic protein production. Plant Biotechnol J 8:564-587.
Grohs B M, Niu Y, Veldhuis L J, Trabelsi S, Garabagi F, Hassell J A, McLean M D and Hall J C (2010) Plant-produced trastuzumab inhibits the growth of HER2 positive cancer cells. J Agric Food Chem 58:10056-10063.
Horsch R. B., Fry J. E., Hoffmann N. L., Eichholtz D., Rogers S. G. and Fraley R. T. 1985. A simple and general method for transferring genes into plants. Science 227: 1229-1231.
Hsieh Y C, Omarov R T and Scholthof H B (2009) Diverse and newly recognized effects associated with short interfering RNA binding site modifications on the Tomato bushy stunt virus p19 silencing suppressor. J Virol 83:2188-2200.
Jin C, Altmann F, Strasser R, Mach L, Schahs M, Kunert R, Rademacher T, Glossl J and Steinkellner H (2008) A plant-derived human monoclonal antibody induces an anti-carbohydrate immune response in rabbits. Glycobiology 18:235-241.
Jovel J, Walker M and Sanfacon H (2011) Salicylic acid-dependent restriction of Tomato ringspot virus spread in tobacco is accompanied by a hypersensitive response, local RNA silencing, and moderate systemic resistance. Mal Plant Microbe Interact 24:706-718.
Kozak M. (1984) Compilation and analysis of sequences upstream from the translational start site in eukaryotic mRNAs. Nucleic Acids Res 12, 857-72.
Lewis G D, Figari I, Fendly B, Wong W L, Carter P, Gorman C and Shepard H M (1993) Differential responses of human tumor cell lines to anti-p185HER2 monoclonal antibodies. Cancer Immunol Immunother 37:255-263.
Li, Q.; Hunt, A. A near-stream element in a plant polyadenylation signal consist of more than six nucleotides. Plant Mol. Biol. 1995, 28, 927-934.
Moffett P (2009) Mechanisms of recognition in dominant R gene mediated resistance. Adv Virus Res 75:1-33.
Makvandi-Nejad S., McLean M. D., Hirama T., Almquist K. C., Mackenzie C. R. and Hall J. C. (2005) Transgenic tobacco plants expressing a dimeric single-chain variable fragment (scfv) antibody against Salmonella enterica serotype Paratyphi B. Transgenic Res 14, 785-92.
Marillonnet, S., Thoeringer, C., Kandzia, R., Klimyuk, V., and Gleba, Y. (2005). Systemic Agrobacterium tumefaciens-mediated transfection of viral replicons for efficient transient expression in plants. Nat. Biotechnol 23, 718-723.
McLean, M. D., Almquist, K. C., Niu, Y., Kimmel, R., Lai, Z., Schreiber, J. R., and Hall, J. C. (2007). A Human Anti-Pseudomonas aeruginosa Serotype O6ad Immunoglobulin G1 Expressed in Transgenic Tobacco Is Capable of Recruiting Immune System Effector Function In Vitro. Antimicrob. Agents Chemother. 51, 3322-3328.
Nakamura, Y. Codon usage database. Available on-line at www.kazusa.or.jp/codon; 2005.
Ohme-Takagi, M.; Taylor, C.; Newman, T.; Green, P. The effect of sequences with high AU content on mRNA stability intobacco. Proc. Natl. Acad. Sci. U.S.A. 1993, 90, 11811-11815.
Olea-Popelka, F., McLean, M. D., Horsman, J., Almquist, K., Brandle, J. E., and Hall, J. C. (2005). Increasing expression of an anti-picloram single-chain variable fragment (ScFv) antibody and resistance to picloram in transgenic tobacco (Nicotiana tabacum). J. Agric. Food Chem 53, 6683-6690.
Park J W, Faure-Rabasse S, Robinson M A, Desvoyes B and Scholthof H B (2004) The multifunctional plant viral suppressor of gene silencing P19 interacts with itself and an RNA binding host protein. Virology 323:49-58.
Qiu W, Park J W and Scholthof H B (2002) Tombusvirus P19-mediated suppression of virus-induced gene silencing is controlled by genetic and dosage features that influence pathogenicity. Mol Plant Microbe Interact 15:269-280.
Reinhart B J, Weinstein E G, Rhoades M W, Bartel B and Bartel D P (2002) MicroRNAs in plants. Genes Dev 16:1616-1626.
Rothnie, H. M. Plant mRNA 3□-end formation. Plant Mol. Biol. 1996, 32, 43-61.
Samac, D. A., Hironaka, C. M., Yallaly, P. E., and Shah, D. M. (1990). Isolation and Characterization of the Genes Encoding Basic and Acidic Chitinase in Arabidopsis thaliana. Plant Physiol 93, 907-914.
Saxena P, Hsieh Y C, Alvarado V Y, Sainsbury F, Saunders K, Lomonossoff G P and Scholthof H B (2011) Improved foreign gene expression in plants using a virus-encoded suppressor of RNA silencing modified to be developmentally harmless. Plant Biotechnol J 9:703-712.
Schiestl M, Stangler T, Torella C, Cepeljnik T, Toll H and Grau R (2011) Acceptable changes in quality attributes of glycosylated biopharmaceuticals. Nature Biotechnology 29:310-312.
Scholthof H B (2006) The Tombusvirus-encoded P19: from irrelevance to elegance. Nat Rev Microbiol 4:405-411.
Scholthof H B, Desvoyes B, Kuecker J and Whitehead E (1999) Biological activity of two tombusvirus proteins translated from nested genes is influenced by dosage control via context-dependent leaky scanning. Mol Plant Microbe In 12:670-679,
Scholthof K B G, Scholthof H B and Jackson A O (1995) The Effect of Defective Interfering Rnas on the Accumulation of Tomato Bushy Stunt Virus Proteins and Implications for Disease Attenuation. Virology 211:324-328.
Shapiro, M. B.; Senapathy, P. RNA splice junctions of different classes of eukaryotes: sequence statistics and functional implications in gene expression. Nucleic Acids Res. 1987, 5, 7155-7174.
Siddiqui S A, Sarmiento C, Truve E, Lehto H and Lehto K (2008) Phenotypes and functional effects caused by various viral RNA silencing suppressors in transgenic Nicotiana benthamiana and N. tabacum. Mol Plant Microbe Interact 21:178-187.
Sourrouille C, Marquet-Blouin E, D'Aoust M A, Kiefer-Meyer M C, Seveno M, Pagny-Salehabadi S, Bardor M, Durambur G, Lerouge P, Vezina L and Gomord V (2008) Down-regulated expression of plant-specific glycoepitopes in alfalfa. Plant Biotechnol J 6:702-721.
Stadlmann J, Pabst M, Kolarich D, Kunert R and Altmann F (2008) Analysis of immunoglobulin glycosylation by LC-ESI-MS of glycopeptides and oligosaccharides. Proteomics 8:2858-2871.
Stevens L H, Stoopen G M, Elbers I J, Molthoff J W, Bakker H A, Lommen A, Bosch D and Jordi W (2000) Effect of climate conditions and plant developmental stage on the stability of antibodies expressed in transgenic tobacco. Plant Physiol 124:173-182.
Strasser R, Stadlmann J, Schahs M, Stiegler G, Quendler H, Mach L, Glossl J, Weterings K, Pabst M and Steinkellner H (2008) Generation of glycoengineered Nicotiana benthamiana for the production of monoclonal antibodies with a homogeneous human-like N-glycan structure. Plant Biotechnol J 6:392-402.
Vezina L P, Faye L, Lerouge P, D'Aoust M A, Marquet-Blouin E, Burel C, Lavoie P O, Bardor M and Gomord V (2009) Transient co-expression for fast and high-yield production of antibodies with human-like N-glycans in plants. Plant Biotechnol J 7:442-455.
Voinnet O, Pinto Y M and Baulcombe D C (1999) Suppression of gene silencing: a general strategy used by diverse DNA and RNA viruses of plants. Proc Natl Acad Sci USA 96:14147-14152.
Voinnet O, Rivas S, Mestre P and Baulcombe D (2003) An enhanced transient expression system in plants based on suppression of gene silencing by the p19 protein of tomato bushy stunt virus. Plant J 33:949-956.
Weber, A., H. Butterweck, U. Mais-Paul, W. Teschner, L. Lei, E. M. Muchitsch, D. Kolarich, F. Altmann, H. J. Ehrlich, and H. P. Schwarz. 2010. Biochemical, molecular and preclinical characterization of a double-virus-reduced human butyrylcholinesterase preparation designed for clinical use. Vox Sang 100:285-97.
Yu D., McLean M. D., Hall J. C. and Ghosh R. (2008) Purification of a human immunoglobulin G1 monoclonal antibody from transgenic tobacco using membrane chromatographic processes. J Chromatogr A 1187, 128-37.
Zeitlin L, Pettitt J, Scully C, Bohorova N, Kim D, Pauly M, Hiatt A, Ngo L, Steinkellner H, Whaley K J and Olinger G G (2011) Enhanced potency of a fucose-free monoclonal antibody being developed as an Ebola virus immunoprotectant. Proc Natl Acad Sci USA 108:20690-20694.
Zheng N, Xia R, Yang C, Yin B, Li Y, Duan C, Liang L, Guo H and Xie Q (2009) Boosted expression of the SARS-CoV nucleocapsid protein in tobacco and its immunogenicity in mice. Vaccine 27:5001-5007.

1. An expression vector comprising
(a) a promoter selected from (i) the 35S promoter of the Cauliflower Mosaic Virus (CaMV) or (ii) the promoter of the ribulose bisphosphate carboxylase (rbc) small subunit gene of Chrysanthemum morifolium,
(b) a 5′ untranslated region (UTR) selected from (i) the 35S 5′ UTR of CaMV or (ii) the 5′ UTR of the rbc small subunit gene of C. morifolium; and
(c) a 3′ UTR and terminator sequence selected from (i) the 3′ UTR and terminator sequence of the nopaline synthase (nos) gene of Agrobacterium, (ii) the 3′ UTR and terminator sequence of the osmotin (osm) gene of Oryza sativa, (iii) the 3′ UTR and terminator sequence from the rbc small subunit gene of C. morifolium or (iv) a truncated version, by 162 bp as defined by a BspEI recognition site, of the 3′ UTR and terminator sequence from the rbc small subunit gene of C. morifolium.

2. The expression vector of claim 1 wherein the vector comprises: the 35S promoter of CaMV, operably linked to the 35S 5′ UTR of CaMV and the 3′ UTR and terminator sequence of the nos gene of Agrobacterium; the 35S promoter of CaMV, operably linked to the 35S 5′ UTR of CaMV and the 3′ UTR and terminator sequence of the osm gene of Oryza sativa; the 35S promoter of CaMV, operably linked to the 35S 5′ UTR of CaMV and the 3′ UTR and terminator sequence of the rbc small subunit gene of C. morifolium; the 35S promoter of CaMV, operably linked to the 35S 5′ UTR of CaMV and a truncated version, by 162 bp as defined by a BspEI recognition site, of the 3′ UTR and terminator sequence from the rbc small subunit gene of C. morifolium; or the promoter of the rbc small subunit gene of C. morifolium, operably linked to the 5′ UTR of the rbc small subunit gene of C. morifolium and the 3′ UTR and terminator sequence of the rbc small subunit gene of C. morifolium.

3. The expression vector of claim 1 wherein the expression vector further comprises a nucleic acid molecule encoding the P19 protein from Tomato Bushy Stunt Virus (TBSV).

4. The expression vector of claim 3 wherein the nucleic acid molecule encoding the P19 protein has the sequence shown in SEQ ID NO:1.

5. The expression vector of claim 1 wherein the expression vector further comprises a nucleic acid sequence encoding a recombinant protein.

6. The expression vector of claim 5 wherein the recombinant protein comprises an antibody or antibody fragment.

7. The expression vector of claim 6 wherein the antibody is trastuzumab or bevacizumab.

8. The expression vector of claim 5 wherein the recombinant protein comprises a therapeutic enzyme.

9. The expression vector of claim 8 wherein the therapeutic enzyme is butyrylcholinesterase.

10. The expression vector of claim 1 further comprising the Arabidopsis heat-shock promoter (Hsp81.1).

11. A method of enhancing the production of a recombinant protein in a plant comprising:
(a) introducing an expression vector according to claim 5 into a plant or plant cell; and
(b) growing the plant or plant cell to obtain a plant that expresses the recombinant protein.

12. A method according to claim 11 wherein the recombinant protein is an antibody or fragment thereof.

13. A method according to claim 12 wherein the antibody or fragment thereof is trastuzumab or bevacizumab.

14. A method according to claim 11 wherein the recombinant protein is a therapeutic enzyme.

15. A method according to claim 14 wherein the therapeutic enzyme is butyrylcholinesterase.

16. A method according to claim 11 further comprising introducing a nucleic acid molecule encoding the P19 protein from TBSV.

17. A method according to claim 16 wherein the nucleic acid molecule encoding the P19 protein has the sequence shown in SEQ ID NO:1.

18. A method according to claim 5 wherein the plant is a tobacco plant.

19. A method according to claim 18 wherein the tobacco plant is N. benthamiana or N. tabacum.

20. A method according to claim 18 wherein the N. tabacum is cv. Little Crittenden.


SEQUENCE LISTING (3 sequences)

SEQ ID NO: 1 - DNA, 519 bp, Nicotiana
atggaaagggctattcagggaaatgatgctagagagcaggctaattctgaaagatgggatggtggatctggtggaactacttctccattcaagcttccagatgagtctccatcttggactgagtggaggcttcataacgatgagactaactccaatcaggataacccactcggattcaaagaatcttggggattcggaaaggttgtgttcaagcgttaccttaggtatgataggactgaggcttcacttcatagggttctcggatcttggactggtgattctgttaactacgctgcttctcgtttttttggattcgatcagatcggatgcacttactctattaggttcaggggagtgtctattactgtttctggtggatctaggactcttcaacacctttgcgagatggctattaggtctaagcaagagcttcttcagcttgctccaattgaggttgagtctaacgtttcaagaggatgtccagaaggtactgagactttcgagaaagaatccgagtga

SEQ ID NO: 2 - protein, 21 aa, Arabidopsis thaliana
MAKTNLFLFLIFSLLLSLSSA

SEQ ID NO: 3 - protein, 28 aa, Homo Sapien
MHSKVTIICIRFLFWFLLLCMLIGKSHT